SEQUENCE LISTING (18 sequences)

SEQ ID NO: 1 - DNA, 23 bp, Artificial Sequence
  Synthetic siCtl (forward)
ccuacaucccgaucgaugaugtt

SEQ ID NO: 2 - DNA, 19 bp, Artificial Sequence
  Synthetic siVDAC1 (forward)
gauacacucagacucuaaa

SEQ ID NO: 3 - DNA, 21 bp, Artificial Sequence
  Synthetic siHIF-1alpha (forward)
cugaugaccagcaacuugatt

SEQ ID NO: 4 - DNA, 21 bp, Artificial Sequence
  Synthetic siHIF-2alpha (forward)
cagcaucuuugauagcagutt

SEQ ID NO: 5 - DNA, 22 bp, Artificial Sequence
  Synthetic GLI1 (forward)
tgcagtaaagccttcagcaatg

SEQ ID NO: 6 - DNA, 20 bp, Artificial Sequence
  Synthetic GLI1 (reverse)
ttttcgcagcgagctaggat

SEQ ID NO: 7 - DNA, 21 bp, Artificial Sequence
  Synthetic IFT20 (forward)
ggtatcgggttgaatatgaag

SEQ ID NO: 8 - DNA, 20 bp, Artificial Sequence
  Synthetic IFT20 (reverse)
gacataggtcattggtcaag

SEQ ID NO: 9 - DNA, 20 bp, Artificial Sequence
  Synthetic PDGFRA (forward)
tcaagttccttcatccattc

SEQ ID NO: 10 - DNA, 21 bp, Artificial Sequence
  Synthetic PDGFRA (reverse)
catccactcaatatcaggaag

SEQ ID NO: 11 - DNA, 18 bp, Artificial Sequence
  Synthetic PRKCA (forward)
ccaaagtgtgtggcaaag

SEQ ID NO: 12 - DNA, 20 bp, Artificial Sequence
  Synthetic PRKCA (reverse)
tcagactggtctatgttagc

SEQ ID NO: 13 - DNA, 20 bp, Artificial Sequence
  Synthetic FZD1 (forward)
actgcattaaatcctgtgtg

SEQ ID NO: 14 - DNA, 20 bp, Artificial Sequence
  Synthetic FZD1 (reverse)
gcttttctcctcttcttcac

SEQ ID NO: 15 - DNA, 20 bp, Artificial Sequence
  Synthetic LGMN (forward)
actatgatgagaagaggtcc

SEQ ID NO: 16 - DNA, 20 bp, Artificial Sequence
  Synthetic LGMN (reverse)
ggtggagattgttttgtttc

SEQ ID NO: 17 - DNA, 20 bp, Artificial Sequence
  Synthetic PDL1 (forward)
atgccccatacaacaaaatc

SEQ ID NO: 18 - DNA, 21 bp, Artificial Sequence
  Synthetic PDL1 (reverse)
gacatgtcagttcatgttcag

(19) United States
(12) Patent Application Publication
(10) Pub. No.: US 2022/0098674 A1
(43) Pub. Date: Mar. 31, 2022

(54) METHODS AND COMPOSITIONS FOR SELECTING A CANCER TREATMENT IN A SUBJECT SUFFERING FROM CANCER

(71) Applicants: INSERM (INSTITUT NATIONAL DE LA SANTÉ ET DR LA RECHERCH MÉDICALE), Paris (FR); CENTRE NATIONAL DE LA RECHERCHE SCIENTIFIQUE - CNRS -, Paris (FR); UNIVERSITE COTE D'AZUR, Nice Cedex 2 (FR)

(72) Inventors: Maeva DUFIES, Nice (FR); Nathalie MAZURE, Tourrette Levens (FR); Lucilla FABBRI, Nice (FR)

(21) Appl. No.: 17/430,134

(22) PCT Filed: Feb. 13, 2020
(86) PCT No.: PCT/EP2020/053812

§ 371 Date: Aug. 11, 2021

(30) Foreign Application Priority Data
Feb. 13, 2019 (EP) 19305179.4
Jun. 14, 2019 (EP) 19305762.7

(51) Int. Cl. C12Q 1/6886

(52) U.S. Cl. CPC C12Q 1/6886; C12Q 2600/158; C12Q 2600/106; C12Q 2600/16

(57) ABSTRACT

The invention relates to methods and compositions for selecting a cancer treatment in a subject suffering from cancer. The inventors have identified a 2-genes signature that determines the presence or absence of the primary cilium in relationship to the presence of VDAC1-AC. The inventors provide a new way to classify patients suffering from cancer and propose potential therapeutic targets linked to metabolism and immunotherapy. In particular, the invention relates to methods for selecting a cancer treatment in a subject suffering from cancer, wherein said method comprises the step of determining, in a biological sample (e.g. tissue biopsy) obtained from said subject, the expression level of GLI family zinc finger 1 (GLI1) and intraflagellar transport 20 (IFT20) and concluding the cancer treatment of said subject.

### FIELD OF THE INVENTION

The invention is in the field of oncology. More particularly, the invention relates to methods and compositions for selecting a cancer treatment in a subject suffering from cancer.

### BACKGROUND OF THE INVENTION

Among the dysfunctions of tumor cells, an abnormality that has recently emerged concerns a decrease or loss of the primary cilium, a small sensory organelle in which many signaling factors are known to be concentrated (1,2). This loss defines cancer as a form of ciliopathy (3-5). The primary cilium is a single protrusion emerging from the apical surface of the cell membrane of nearly all mammalian cells during interphase. It senses external signals from the microenvironment and initiates corresponding signaling cascades to the rest of the cell, such as the Hedgehog (Hh) and Wingless (Wnt) pathways (6-10). Its structure is built of a microtubule-based axoneme, which confers mechanical strength and guides the transport of molecules. Any defects in the structure, the activity or the function of the primary cilium will affect multiple systems, the consequences of which can be devastating or lifethreatening. There are many phenotypes that are regularly associated with ciliopathies, including renal diseases (11), with the kidneys being among the organs that are most highly affected. A spectrum of renal diseases has been described as a feature of several ciliopathic syndromes and includes a morphologically heterogeneous group of disorders that have been classified as polycystic, renal medullary cystic disease, cystic renal dysplasia and, more recently, renal cell carcinoma (12-14). The von Hippel-Lindau (VHL) protein encoded by a known tumor suppressor gene, has been shown to maintain cilia (12,13). Mutations or deletions in the VHL gene, in addition to methylation, characterize: (i) a rare hereditary tumor disease resulting from germline alterations of the VHL gene (15) and (ii) sporadic clear cell renal cell carcinoma (ccRCC) lacking cilia (16). The pVHL protein, a component of an E3 ubiquitin ligase complex, ubiquitylates HIFs and targets them for degradation by the proteasome (17). Interestingly, ccRCCs deficient in pVHL cluster into tumors that express either both HIF-1α and -2α or HIF-2α only. The voltage-dependent anion channel 1 (VDAC1) is the most abundant protein of the mitochondrial outer membrane. VDAC1 has fundamental functions in regulating energy production, in calcium signaling and in promoting apoptotic signaling (18,19). A strong relationship between VDAC and Hexokinase, the first enzyme of glycolysis, confirms the interconnection between the regulation of glycolysis and mitochondrial respiration. The inventors have further described this role by studying VDAC1 under hypoxic conditions, in a HIF-1-dependent manner, and showed that the presence of a cleaved form of VDAC1 (VDAC1-AC) plays a novel role in promoting resistance to apoptosis, in increasing metabolism and thus in cancer cell survival (20,21). They characterized this cleavage by the asparagine endopeptidase (AEP), also known as LGMN, at the asparagine 214 to produce VDAC1-AC (22). They then showed that knockout of Vdac1 expressing oncogenic RAS in murine embryonic fibroblasts (MEFs) potentiates tumor development in mice by promoting metabolic reprogramming, accelerating vascular destabilization and inflammation (22). Finally, a new function for VDAC1 has recently been discovered. Centrosomal VDAC1 has been predominantly localized to the mother centriole and down-regulation of VDAC1 led to inappropriate ciliogenesis (23,24). The inventors showed that VDAC1 and VDAC3 both negatively modulate primary cilium but with non-redondant functions. VDAC1 not only regulates apoptosis and metabolism, but it may also control cell cycle/signaling pathways by controlling primary cilium. The inventors therefore sought to explore the function of mitochondrial VDAC1-AC in a ccRCC cells and patient context, a rare cancer model where ciliopathy is concomittant with HIF stabilization. Moreover, they hypothesized that mitochondrial VDAC1 could also control ciliogenesis. Interestingly, the inventors identified a new group of ccRCC patients in which the primary cilium is re-expressed, in the absence of the cleaved form of VDAC1, giving rise to increased tumor aggressiveness.

Only some patients respond to the current treatment. Accordingly, identify new biomarkers to direct patients to appropriate treatment is needed to ensure the best medical care.

### SUMMARY OF THE INVENTION

The invention relates to methods for selecting a cancer treatment in a subject suffering from cancer, wherein said method comprises the step of determining, in a biological sample obtained from said subject, the expression level of GLI family zinc finger 1 (GLI1) and intraflagellar transport 20 (IFT20), and optionally platelet derived growth factor receptor alpha (PDGFRA), protein kinase C alpha (PRKCA) and frizzled class receptor 1 (FZD1) and concluding the cancer treatment of said subject. In particular, the present invention is defined by the claims.

In a first aspect, the invention relates to a method for selecting a cancer treatment in a subject suffering from a cancer wherein said method comprises the step of:
(i) Determining in a biological sample obtained from said subject the expression level of GLI family zinc finger 1 (GLI1) and intraflagellar transport 20 (IFT20)
(ii) Comparing the expression levels quantified at step i) with their predetermined references values and
(iii) Concluding that the subject will be treated with at least one therapeutic target linked to metabolism and/or immunotherapy, preferably with an immune checkpoint inhibitor or an inhibitor of lactate dehydrogenase (LDH) or an inhibitor of glycolysis, if the expression level of GLI1 is higher than its predetermined reference value 1 (PRV1) and if the expression level of IFT20 is higher than its predetermined reference value 2 (PRV2).

In a second aspect, the invention relates to a method for selecting a cancer treatment in a subject suffering from a cancer wherein said method comprises the step of:
(i) Determining in a biological sample obtained from said subject the expression level of GLI family zinc finger 1 (GLI1) and intraflagellar transport 20 (IFT20)
(ii) Comparing the expression levels quantified at step i) with their predetermined references values and
(iii) Concluding that the subject will be treated with a current treatment, preferably a tyrosine kinase inhibitor, if
   a) the expression level of GLI1 is lower than its PRV1 and the expression level of IFT20 is higher than its PRV2, or
   b) the expression level of GLI1 is higher than its PRV1 and the expression level of IFT20 is lower than its PRV2, or
   c) the expression level of GLI1 is lower than its PRV1 and the expression level of IFT20 is lower than its PRV2.

In another aspect, the invention relates to a method for selecting patients wherein the subject will be treated with an immune checkpoint inhibitor in combination with an inhibitor of lactate dehydrogenase (LDH) or an inhibitor of glycolysis if the expression level of GLI1 is higher than its predetermined reference value 1 (PRV1) and if the expression level of IFT20 is higher than its predetermined reference value 2 (PRV2).

In another embodiment, the invention relates to a method for treating a subject suffering from a cancer, wherein said method comprises the step of:

(i) Determining in a biological sample obtained from said subject the expression level of GLI1 and IFT20
(ii) Comparing the expression levels quantified at step i) with their predetermined references values and
(iii) Administering to said subject a therapeutically effective amount of at least one therapeutic target linked to metabolism and/or immunotherapy, preferably an immune checkpoint inhibitor or an inhibitor of lactate dehydrogenase (LDH) or an inhibitor of glycolysis, if the expression level of GLI1 is higher than its predetermined reference value 1 (PRV1) and if the expression the expression level of IFT20 is higher than its predetermined references values 2 (PRV2).

In another embodiment, the invention relates to a method for treating a subject suffering from a cancer, wherein said method comprises the step of:
(i) Determining in a biological sample obtained from said subject the expression level of GLI1 and IFT20
(ii) Comparing the expression levels quantified at step i) with their predetermined references values and
(iii) Administering to said subject a therapeutically effective amount of a current treatment, preferably a tyrosine kinase inhibito, if:
   a) the expression level of GLI1 is lower than its PRV1 and the expression level of IFT20 is higher than its PRV2, or
   b) the expression level of GLI1 is higher than its PRV1 and the expression level of IFT20 is lower than its PRV2, or
   c) the expression level of GLI1 is lower than its PRV1 and the expression level of IFT20 is lower than its PRV2.

In another embodiment, the step (iii) of the method for treating patients consists of administering to said subject a therapeutically effective amount of an immune checkpoint inhibitor in combination with an inhibitor of lactate dehydrogenase (LDH) or an inhibitor of glycolysis if the expression level of GLI1 is higher than its predetermined reference value 1 (PRV1) and if the expression level of IFT20 is higher than its predetermined reference value 2 (PRV2).

Optionally, the invention relates to the method wherein the cancer is a ciliopathy.

Preferably, the cancer is a kidney cancer. More preferably the cancer is a Clear cell renal cell carcinoma (ccRCC).

In an embodiment, the tyrosine kinase inhibitor is sunitinib.

In an embodiment, the immune checkpoint inhibitor is anti-PD-L1 or anti-PD-1.

Optionally, the immune checkpoint inhibitor is atezolizumab.

Alternatively, the immune checkpoint inhibitor nivolumab.

In an embodiment, the inhibitor of lactate dehydrogenase (LDH) is selected from dichloroacetate (DCA), FX11, AZD-3965.

In an embodiment, the inhibitor of glycolysis is 3-bromopyruvate

### DETAILED DESCRIPTION OF THE INVENTION

The inventors have surprisingly demonstrated that VDAC1-AC controls both metabolism and resorption of the primary cilium in the HIF-1-dependent model of ccRCC but independently of HIF-2. Using cohorts of clear cell Renal Cell Carcinoma (ccRCC) patients, the inventors showed that three patients groups presented ciliopathy correlated with the presence of VDAC1-AC, whereas two VDAC1-AC lacking groups expressed the primary cilium in combination with maintenance of glycolysis, an EMT signature and more aggressive tumor progression. The inventors provide a new way to classify ccRCC patients and propose potential therapeutic targets linked to metabolism and immunotherapy.

Using a 498 patient cohort of prostate cancer (PCa) the inventors have also demonstrated that a small group of patients is re-expressing the primary cilium (GLI+/IFT20+) after the loss of this primary cilium. The PCa is, as the ccRCC, a form of ciliopathy.

Firstly, inventors have identified a 2-genes signature that determines tumor aggressiveness. Patients who express a level of GLI family zinc finger 1 (GLI1) and intraflagellar transport 20 (IFT20) both at a higher level than that of predetermined reference value present a poor response to current treatment such as chemotherapy or radiotherapy and a better response to immunotherapy. Thus, the present 2-genes signature enables to suitably select the cancer treatment given as first-line treatment in a subject suffering from a cancer.

Moreover, inventors have identified that this 2-genes signature determines the presence or absence of the primary cilium in relationship to the presence of VDAC1-AC. Thus, the inventors identified subjects who express the primary cilium and display higher tumor aggressiveness.

In a first aspect, the invention relates to a method for selecting a cancer treatment in a subject suffering from a cancer, wherein said method comprises the step of:
(i) Determining in a biological sample obtained from said subject the expression level of GLI family zinc finger 1 (GLI1) and intraflagellar transport 20 (IFT20),
(ii) Comparing the expression levels quantified at step i) with their predetermined references values and
(iii) Concluding that the subject will be treated with:
   a) At least one therapeutic target linked to metabolism and/or immunotherapy, preferably an immune checkpoint inhibitor or an inhibitor of lactate dehydrogenase (LDH) or an inhibitor of glycolysis, if the expression level of GLI1 is higher than its predetermined reference value 1 (PRV1) and if the expression level of IFT20 is higher than its predetermined reference value 2 (PRV2), or
   b) A current treatment, preferably, a tyrosine kinase inhibitor, if the expression level of GLI1 is lower than its PRV1 and if the expression level of IFT20 is higher than its PRV2, or
   c) A current treatment, preferably, a tyrosine kinase inhibitor, if the expression level of GLI1 is higher than its PRV1 and if the expression level of IFT20 is lower than its PRV2, or
   d) A current treatment, preferably, a tyrosine kinase inhibitor, if the expression level of GLI1 is lower than its PRV1 and if the expression level of IFT20 is lower than its PRV2.

The invention optionally relates to a method for selecting a cancer treatment in a subject suffering from a cancer, wherein said method comprises the step of:
(i) Determining in a biological sample obtained from said subject the expression level of GLI family zinc finger 1 (GLI1), intraflagellar transport 20 (IFT20), platelet derived growth factor receptor alpha (PDGFRA), protein kinase C alpha (PRKCA) and frizzled class receptor 1 (FZD1)
(ii) Comparing the expression levels quantified at step i) with their predetermined references values and
(iii) Concluding that the subject will be treated with:
   a) At least one therapeutic target linked to metabolism and/or immunotherapy, preferably an immune checkpoint inhibitor or an inhibitor of lactate dehydrogenase (LDH) or an inhibitor of glycolysis, if the expression level of GLI1 is higher than its predetermined reference value 1 (PRV1), if the expression level of IFT20 is higher than its predetermined reference value 2 (PRV2), if the expression level of PDGFRA is higher than its predetermined reference value 3 (PRV3), if the expression level of PRKCA is higher than its predetermined reference value 4 (PRV4) and if the expression level of FZD1 is lower than its predetermined reference value 5 (PRV5), or
   b) At least one therapeutic target linked to metabolism and/or immunotherapy, preferably an immune checkpoint inhibitor or an inhibitor of lactate dehydrogenase or an inhibitor of glycolysis if the expression level of GLI1 is higher than its PRV1, if the expression level of IFT20 is higher than its PRV2, if the expression level of PDGFRA is lower than its PRV3, if the expression level of PRKCA is lower than its PRV4 and if the expression level of FZD1 is higher than its PRV5, or
   c) A current treatment, preferably, a tyrosine kinase inhibitor, if the expression level of GLI1 is lower than its PRV1, if the expression level of IFT20 is higher than its PRV2, if the expression level of PDGFRA is lower than its PRV3, if the expression level of PRKCA is lower or higher than its PRV4 and if the expression level of FZD1 is lower than its PRV5, or
   d) A current treatment, preferably, a tyrosine kinase inhibitor, if the expression level of GLI1 is higher than its PRV1, if the expression level of IFT20 is lower than its PRV2, if the expression level of PDGFRA is lower than its PRV3, if the expression level of PRKCA is lower or higher than its PRV4 and if the expression level of FZD1 is lower or higher than its PRV5, or
   e) A current treatment, preferably, a tyrosine kinase inhibitor, if the expression level of GLI1 is lower than its PRV1, if the expression level of IFT20 is lower than its PRV2, if the expression level of PDGFRA is lower than its PRV3, if the expression level of PRKCA is lower or higher than its PRV4 and if the expression level of FZD1 is lower than its PRV5.

As used herein, the term “selecting” refers to choose in preference to another or others, to pick out, to make a choice. In the context of the invention, the term “selecting” refers to choose from several treatments, to pick out, to make a choice between several treatments, notably treatments that are administrated as first-line treatment. More particularly, the invention is suitable to select an appropriate treatment such as use of an immune checkpoint inhibitor, an inhibitor of lactate dehydrogenase (LDH) or an inhibitor of glycolysis; or a tyrosine kinase inhibitor. Particularly, the invention is suitable to select an appropriate treatment such as either use of an immune checkpoint inhibitor in combination or not with an inhibitor of lactate dehydrogenase (LDH) or an inhibitor of glycolysis; or use of a tyrosine kinase inhibitor.

The invention also relates to a method for adapting a cancer treatment in a subject suffering from cancer, wherein said method comprises the step of:
(i) Determining in a biological sample obtained from said subject the expression level of GLI1 and IFT20,
(ii) Comparing the expression levels quantified at step i) with their predetermined references values and
(iii) Concluding that the subject will be treated with:
   a) At least one therapeutic target linked to metabolism and/or immunotherapy, preferably an immune checkpoint inhibitor or an inhibitor of lactate dehydrogenase (LDH) or an inhibitor of glycolysis, if the expression level of GLI1 is higher than its predetermined reference value 1 (PRV1) and if the expression level of IFT20 is higher than its predetermined reference value 2 (PRV2), or
   b) A current treatment, preferably, a tyrosine kinase inhibitor, if the expression level of GLI1 is lower than its PRV1 and if the expression level of IFT20 is higher than its PRV2,
   c) A current treatment, preferably, a tyrosine kinase inhibitor, if the expression level of GLI1 is higher than its PRV1 and if the expression level of IFT20 is lower than its PRV2, or
   d) A current treatment, preferably, a tyrosine kinase inhibitor, if the expression level of GLI1 is lower than its PRV1 and if the expression level of IFT20 is lower than its PRV2

The invention optionally relates to a method for adapting a cancer treatment in a subject suffering from cancer, wherein said method comprises the step of:
(i) Determining in a biological sample obtained from said subject the expression level of GLI1, IFT20, PDGFRA, PRKCA and FZD1
(ii) Comparing the expression levels quantified at step i) with their predetermined references values and
(iii) Concluding that the subject will be treated with:
   a) At least one therapeutic target linked to metabolism and/or immunotherapy, preferably an immune checkpoint inhibitor or an inhibitor of lactate dehydrogenase (LDH) or an inhibitor of glycolysis if the expression level of GLI1 is higher than its predetermined reference value 1 (PRV1), if the expression level of IFT20 is higher than its predetermined reference value 2 (PRV2), if the expression level of PDGFRA is higher than its predetermined reference value 3 (PRV3), if the expression level of PRKCA is higher than its predetermined reference value 4 (PRV4) and if the expression level of FZD1 is lower than its predetermined reference value 5 (PRV5), or
   b) At least one therapeutic target linked to metabolism and/or immunotherapy, preferably an immune checkpoint inhibitor or an inhibitor of lactate dehydrogenase or an inhibitor of glycolysis if the expression level of GLI1 is higher than its PRV1, if the expression level of IFT20 is higher than its PRV2, if the expression level of PDGFRA is lower than its PRV3, if the expression level of PRKCA is lower than its PRV4 and if the expression level of FZD1 is higher than its PRV5, or
   c) A current treatment, preferably, a tyrosine kinase inhibitor, if the expression level of GLI1 is lower than its PRV1, if the expression level of IFT20 is higher than its PRV2, if the expression level of PDGFRA is lower than its PRV3, if the expression level of PRKCA is lower or higher than its PRV4 and if the expression level of FZD1 is lower than its PRV5, or
   d) A current treatment, preferably, a tyrosine kinase inhibitor, if the expression level of GLI1 is higher than its PRV1, if the expression level of IFT20 is lower than its PRV2, if the expression level of PDGFRA is lower than its PRV3, if the expression level of PRKCA is lower or higher than its PRV4 and if the expression level of FZD1 is lower or higher than its PRV5, or
   e) A current treatment, preferably, a tyrosine kinase inhibitor, if the expression level of GLI1 is lower than its PRV1, if the expression level of IFT20 is lower than its PRV2, if the expression level of PDGFRA is lower than its PRV3, if the expression level of PRKCA is lower or higher than its PRV4 and if the expression level of FZD1 is lower than its PRV5.

As used herein, the term “adapting” refers to adjust, to modify, to suit different conditions or uses. In the context of the invention, the term “adapting” refers to adjust, to modify, to suit different treatments. More particularly, the invention is suitable to adapt an appropriate treatment such as use of an immune checkpoint inhibitor or an inhibitor of lactate dehydrogenase (LDH) or an inhibitor of glycolysis or a tyrosine kinase inhibitor.

Particularly, the invention is suitable to adapt an appropriate treatment such as either use of an immune checkpoint inhibitor in combination or not with an inhibitor of lactate dehydrogenase (LDH) or an inhibitor of glycolysis; or use of a tyrosine kinase inhibitor.

As used herein, the term “subject” denotes a mammal, such as a rodent, a feline, a canine, and a primate. Particularly, the subject according to the invention is a human. More particularly, the subject according to the invention has or is susceptible to have cancer.

As used herein, the term “subject” encompasses “patient”.

The terms “cancer” has its general meaning in the art and refers to a group of diseases involving abnormal cell growth with the potential to invade or spread to other parts of the body. The term “cancer” further encompasses both primary and metastatic cancers. Examples of cancers that may treated by methods and compositions of the invention include, but are not limited to, cancer cells from the bladder, blood, bone, bone marrow, brain, breast, colon, esophagus, gastrointestinal, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non encapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brennertumor, malignant; phyllodestumor, malignant; synovial sarcoma; mesothelioma, malignant; dysgerminoma; embryonal carcinoma; teratoma, malignant; strumaovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblasticodontosarcoma; ameloblastoma, malignant; ameloblasticfibrosarcoma; pinealoma, malignant; chordoma; glioma, malignant; ependymoma; ependymoma; protoplasmic astrocytoma; fibrillary astrocytoma; astroblastoma; medulloblastoma, glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocyticleukemia; mast cell leukemia; megakaryoblasticleukemia; myeloid sarcoma; and hairy cell leukemia.

In one embodiment, the subject has or is susceptible to have kidney cancer.

As used herein, the term “kidney cancer” has its general meaning in the art and refers to a cancer that has arisen from the kidney. In some embodiments, the kidney cancer is a renal cell carcinoma (RCC). The term “renal cell cancer” or “renal cell carcinoma” (RCC), as used herein, refers to cancer which originates in the lining of the proximal convoluted tubule. More specifically, RCC encompasses several relatively common histologic subtypes: clear cell renal cell carcinoma, papillary (chromophil), chromophobe, collecting duct carcinoma (CDC), and medullary carcinoma. Clear cell renal cell carcinoma (ccRCC) is the most common subtype of RCC. In a particular embodiment, the subject has or is susceptible to clear cell Renal Cell Carcinoma (ccRCC).

In one embodiment, the subject has or is susceptible to suffer from ciliopathy. In the case wherein the cancer is a ciliopathy, the inventors have demonstrated that patients, who present a tumor in which the expression level of GLI1 is higher than its predetermined reference value 1 (PRV1) and the expression level of IFT20 is higher than its predetermined reference value 2 (PRV2), arez suffering of a cancer in which the primary cilium is re-expressed.

As used herein, the terms “ciliopathy”, “ciliopathy disorder”, “ciliopathy disease” and “ciliopathic disease” are used interchangeably and refer to those genetic disorders of the cellular cilia, the cilia anchoring structures, the basal bodies, and/or ciliary function. Said ciliopathy may be characterized by ataxia, intellectual deficiency, visual dysfunction, male infertility, kidney dysfunction, liver dysfunction, skeletal dysplasia, olfactory dysfunction, encephalopathy or their combinations. Examples of such disorder include, but are not limited to, Alstrom Syndrome, Bardet-Biedl Syndrome (BBS) (e.g., BBS1, BBS2, BBS4, BBS5, BBS7, BBS9, BBS10, BBS12, ARL6, MKKS, TTC8, TRIM32), Joubert Syndrome, Meckel-Gruber syndrome, Nephronophthisis, Oral-facial-digital syndrome 1 (OFD1), Senior-Loken Syndrome, kidney cancer, Polycystic kidney disease, Polycystic liver disease, primary ciliary dyskinsesia, asphyxiating thoracic dysplasia, Marden-Walker syndrome, situs inversus/Isomerism, retinal degeneration, cerebello-oculo-renal syndrome, Ellis-van Creveld syndrome, Jeune asphyxiating thoracic dystrophy, Leber congenital maurosis and their combinations.

As used herein, the term “biological sample” refers to any sample obtained from a subject, such as a serum sample, a plasma sample, a urine sample, a blood sample, a lymph sample, or a tissue biopsy. In a particular embodiment, the biological sample is a tissue biopsy.

As used herein, the term “tissue”, when used in reference to a part of a body or of an organ, generally refers to an aggregation or collection of morphologically similar cells and associated accessory and support cells and intercellular matter, including extracellular matrix material, vascular supply, and fluids, acting together to perform specific functions in the body. There are generally four basic types of tissue in animals and humans including muscle, nerve, epithelial, and connective tissues.

In some embodiments, when the subject suffers from a cancer, the tissue sample is a tumor tissue sample. As used herein, the term “tumor tissue sample” means any tissue tumor sample derived from the subject. Said tissue sample is obtained for the purpose of the in vitro evaluation. In some embodiments, the tumor sample may result from the tumor resected from the subject. In some embodiments, the tumor sample may result from a biopsy performed in the primary tumour of the subject or performed in metastatic sample distant from the primary tumor of the subject. In some embodiments, the tumor tissue sample encompasses a global primary tumor (as a whole), a tissue sample from the center of the tumor, a tumor tissue sample collected prior surgery (for follow-up of subjects after treatment for example), and a distant metastasis. The tumor tissue sample can, of course, be subjected to a variety of well-known post-collection preparative and storage techniques (e.g., fixation, storage, freezing, etc.). The sample can be fresh, frozen, fixed (e.g., formalin fixed), or embedded (e.g., paraffin embedded.

As used herein, the term “gene” has its general meaning in the art and refers a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed.

In the present specification, the name of each of the genes of interest refers to the internationally recognised name of the corresponding gene, as found in internationally recognised gene sequences and protein sequences databases, in particular in the database from the HUGO Gene Nomenclature Committee, that is available notably at the following Internet address: https://www.genenames.org/. In the present specification, the name of each of the various biological markers of interest may also refer to the internationally recognised name of the corresponding gene, as found in the internationally recognised gene sequences and protein sequences databases ENTREZ ID, Genbank, TrEMBL or ENSEMBL. Through these internationally recognised sequence databases, the nucleic acid sequences corresponding to each of the gene of interest described herein may be retrieved by the one skilled in the art. (see Table A).

TABLE A
the genes names of the present invention

| Name | Symbol | ENTREZ ID |
|---|---|---|
| GLI family zinc | GLI1 | 2735 |
| finger 1 | | |
| intraflagellar | IFT20 | 90410 |
| transport 20 | | |
| platelet derived | PDGFRA | 5156 |
| growth factor | | |
| receptor alpha | | |
| protein kinase C | PRKCA | 5578 |
| alpha | | |
| frizzled class | FZD1 | 8321 |
| receptor 1 | | |

In some embodiments, the expression level of 1, 2, 3, 4 and/or 5, genes is determined. In some embodiments, the method of the present invention further comprises determining the expression level of at least one gene selected from the group consisting of GLI1, IFT20, PDGFRA, PRKCA, FZD1.

According to the present invention, the predetermined references values are determined from reference samples wherein the expression level of the gene(s) was (were) determined and adjusted. Typically, a set of reference samples characterized using both gene expression and another measurement technique such as immunohistochemistry, flow cytometry, or RNA can be used for defining the panel of the predetermined reference value. Mixtures of known cellular proportions also can be suitable for determining the predetermined reference values.

As used herein, the term “control sample” refers to a tissue or celles, for example a kidney tissue or cells, from a healthy subject, or to a healthy tissue of the subject. The control sample may also refer to:

i. a positive control sample indicative of the amount and/or expression level of said at least one gene in a subject suffering from cancer, preferably kidney cancer, with poor prognosis;

ii. a negative control sample indicative of the amount and/or expression level of said at least one gene in a healthy individual or in a healthy tissue of the subject

In some embodiments, the method of the present invention comprises the step of comparing the determined expression level of GLI1 with a predetermined reference value 1 (PRV1). A used herein, “PRV1” refers to the average expression level of GLI1 in control sample.

In one embodiment, the expression level of GLI1⁺ is detected.

As used herein, the term “GLI1⁺” refers to an expression level of GLI1 which is higher than its PRV1. In a particular embodiment the expression level of GLI1⁺ is compared with its PRV1.

In one embodiment, the expression level of GLI1⁻ is detected.

As used herein, the term “GLI1⁻” refers to an expression level of GLI1 which is lower than its PRV1. In a particular embodiment the expression level of GLI1⁻ is compared with its PRV1.

In some embodiments, the method of the present invention comprises the step of comparing the determined expression level of IFT20 with a predetermined reference value 2 (PRV2). A used herein, “PRV2” refers to the average expression level of IFT20 in control sample.

In one embodiment, the expression level of IFT20⁺ is detected.

As used herein, the term “IFT20⁺” refers to an expression level of IFT20 which is higher than its PRV2. In a particular embodiment the expression level of IFT20⁺ is compared with its PRV2.

In one embodiment, the expression level of IFT20⁻ is detected.

As used herein, the term “IFT20⁻” refers to an expression level of IFT20 which is lower than its PRV2. In a particular embodiment the expression level of IFT20⁻ is compared with its PRV2.

In some embodiments, the method of the present invention comprises the step of comparing the determined expression level of PDGFRA with a predetermined reference value 3 (PRV3). As used herein, “PRV3” refers to the average expression level of PDGFRA in control sample.

In one embodiment, the expression level of PDGFRA⁺ is detected.

As used herein, the term “PDGFRA⁺” refers to an expression level of PDGFRA which is higher than its PRV3. In a particular embodiment the expression level of PDGFRA⁺ is compared with its PRV3.

In one embodiment, the expression level of PDGFRA⁻ is detected.

As used herein, the term “PDGFRA⁻” refers to an expression level of PDGFRA which is lower than its PRV3. In a particular embodiment the expression level of PDGFRA⁻ is compared with its PRV3.

In some embodiments, the method of the present invention comprises the step of comparing the determined expression level of PRKCA with a predetermined reference value 4 (PRV4). As used herein, “PRV4” refers to the average expression level of PRKCA in control sample.

In one embodiment, the expression level of PRKCA⁺ is detected.

As used herein, the term “PRKCA⁺” refers to an expression level of PRKCA which is higher than its PRV4. In a particular embodiment the expression level of PRKCA⁺ is compared with its PRV4.

In one embodiment, the expression level of PRKCA⁻ is detected.

As used herein, the term “PRKCA⁻” refers to an expression level of PRKCA which is lower than its PRV4. In a particular embodiment the expression level of PRKCA⁻ is compared with its PRV4.

In some embodiments, the method of the present invention comprises the step of comparing the determined expression level of FZD1 with a predetermined reference value 5 (PRV5). As used herein, “PRV5” refers to the average of the expression level of FZD1 in control sample.

In one embodiment, the expression level of FZD1⁺ is detected.

As used herein, the term “FZD1⁺” refers to an expression level of FZD1 which is higher than its PRV5. In a particular embodiment the expression level of FZD1⁺ is compared with its PRV5.

In one embodiment, the expression level of FZD1⁻ is detected.

As used herein, the term “FZD1⁻” refers to an expression level of FZD1 which is lower than its PRV5. In a particular embodiment the expression level of FZD1⁻ is compared with its PRV5.

As used herein, the terms “PC+ subject” or “PC+ patient” refer to a subject with primary cilia and with the GLI1⁺/IFT20⁺/PDGFRA⁺/PRKCA⁺/FZD1⁻ signatures or with the GLI1⁺/IFT20⁺/PDGFRA⁻/PRKCA⁻/FZD1⁺ signatures.

As used herein, the terms “PC− subject” or “PC− patient” refer to a subject without a primary cilia and with the GLI1⁺/IFT20⁻/PDGFRA⁻/PRKCA^{±}/FZD1^{±} signatures, GLI1⁻/IFT20⁺/PDGFRA⁻/PRKCA^{±}/FZD1⁻ signatures, GLI1⁻/IFT20⁻/PDGFRA⁻/PRKCA^{±}/FZD1⁻ signatures.

In some embodiments, the method of the present invention comprises the step of comparing the determined expression level of GLI1 with a predetermined reference value 1 (PRV1) and the step of comparing the determined expression level of IFT20 with a predetermined reference value 2 (PRV2).

In some embodiments, the method of the present invention comprises the step of comparing the determined expression level of GLI1 with a predetermined reference value 1 (PRV1), the step of comparing the determined expression level of IFT20 with a predetermined reference value 2 (PRV2), the step of comparing the determined expression level of PDGFRA with a predetermined reference value 3 (PRV3), the step of comparing the determined expression level of PRKCA with a predetermined reference value 4 (PRV4) and the step of comparing the determined expression level of FZD1 with a predetermined reference value 5 (PRV5).

In a particular embodiment, the expression level of GLI1 and IFT20 is determined.

In a particular embodiment, the expression level of GLI1⁺ and IFT20⁺ is detected.

In a particular embodiment, the expression level of GLI1⁺ and IFT20⁻ is detected.

In a particular embodiment, the expression level of GLI1⁻ and IFT20⁻ is detected.

In a particular embodiment, the expression level of GLI1⁻ and IFT20⁺ is detected.

In a particular embodiment, the expression level of GLI1, IFT20, PDGFRA, PRKCA and FZD1 is determined.

In a particular embodiment, the expression level of GLI1⁺, IFT20⁺, PDGFRA⁺, PRKCA⁺, FZD1⁻ is detected.

In a particular embodiment, the expression level of GLI1⁺, IFT20⁺, PDGFRA⁻, PRKCA⁻, FZD1⁺ is detected.

In a particular embodiment, the expression level of GLI1⁺, IFT20⁻, PDGFRA⁻, PRKCA^{±}, FZD1^{±} is detected.

In a particular embodiment, the expression level of GLI1⁻, IFT20⁻, PDGFRA⁻, PRKCA^{±}, FZD1⁻ is detected.

In a particular embodiment, the expression level of GLI1⁻, IFT20⁺, PDGFRA⁻, PRKCA^{±}, FZD1⁻ is detected.

As used herein, the term “expression level” refers to the expression level of GLI1, IFT20 and optionally PDGFRA, PRKCA, FZD1. In particular, it refers to the expression level of GLI1 and/or the expression level of IFT20. Typically, the expression level of the GLI1 gene, IFT20 gene, PDGFRA gene, PRKCA gene or FZD1 gene, in particular GLI1 gene or IFT20 gene may be determined by any technology known by a person skilled in the art. In particular, each gene expression level may be measured at the genomic and/or nucleic and/or protein level. In a particular embodiment, the expression level of gene is determined by measuring the amount of nucleic acid transcripts of each gene. In another embodiment, the expression level is determined by measuring the amount of each gene corresponding protein. The amount of nucleic acid transcripts can be measured by any technology known by a man skilled in the art.

In particular, the measure may be carried out directly on an extracted messenger RNA (mRNA) sample, or on retrotranscribed complementary DNA (cDNA) prepared from extracted mRNA by technologies well-known in the art. Typically, the expression level of a gene is determined by determining the quantity of mRNA. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e.g., Northern blot analysis, in situ hybridization) and/or amplification (e.g., RT-PCR). Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In some embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antl1ranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfor1ic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6dicl1lorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6, 130, 101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can be detected as colored light of a specific wavelength or fluorescence. Semiconductor nanocrystals with different spectral characteristics are described in e.g., U.S. Pat. No. 6,602,671. Semiconductor nanocrystals that can be coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can be produced that are identifiable based on their different spectral characteristics. For example, semiconductor nanocrystals can be produced that emit light of different colors based on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlshad, Calif.).

Additional labels include, for example, radioisotopes (such as ³H), metal chelates such as DOTA and DPTA chelates of radioactive or paramagnetic metal ions like Gd3+, and liposomes.

Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can be used in a metallographic detection scheme. For example, silver in situ hybridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. .1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/0117153.

It will be appreciated by those of skill in the art that by appropriately selecting labelled probe-specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can be labelled with a first hapten, such as biotin, while a second probe that corresponds to a second target sequence can be labelled with a second hapten, such as DNP. Following exposure of the sample to the probes, the bound probes can be detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In some embodiments, the methods of the invention comprise the steps of providing total RNAs extracted from cumulus cells and subjecting the RNAs to amplification and hybridization to specific probes, more particularly by means of a quantitative or semi-quantitative RT-PCR.

In some embodiments, the level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In some embodiments, the nCounter® Analysis system is used to detect intrinsic gene expression. The basis of the nCounter® Analysis system is the unique code assigned to each nucleic acid target to be assayed (International Patent Application Publication No. WO 08/124847, U.S. Pat. No. 8,415,102 and Geiss et al. Nature Biotechnology. 2008. 26(3): 317-325; the contents of which are each incorporated herein by reference in their entireties). The code is composed of an ordered series of colored fluorescent spots which create a unique barcode for each target to be assayed. A pair of probes is designed for each DNA or RNA target, a biotinylated capture probe and a reporter probe carrying the fluorescent barcode. This system is also referred to, herein, as the nanoreporter code system. Specific reporter and capture probes are synthesized for each target. The reporter probe can comprise at a least a first label attachment region to which are attached one or more label monomers that emit light constituting a first signal; at least a second label attachment region, which is non-over-lapping with the first label attachment region, to which are attached one or more label monomers that emit light constituting a second signal; and a first target-specific sequence. Preferably, each sequence specific reporter probe comprises a target specific sequence capable of hybridizing to no more than one gene and optionally comprises at least three, or at least four label attachment regions, said attachment regions comprising one or more label monomers that emit light, constituting at least a third signal, or at least a fourth signal, respectively. The capture probe can comprise a second target-specific sequence; and a first affinity tag. In some embodiments, the capture probe can also comprise one or more label attachment regions. Preferably, the first target-specific sequence of the reporter probe and the second target-specific sequence of the capture probe hybridize to different regions of the same gene to be detected. Reporter and capture probes are all pooled into a single hybridization mixture, the “probe library”. The relative abundance of each target is measured in a single multiplexed hybridization reaction. The method comprises contacting the tumor tissue sample with a probe library, such that the presence of the target in the sample creates a probe pair-target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies). Purified reactions are typically deposited by the Prep Station into individual flow cells of a sample cartridge, bound to a streptavidin-coated surface via the capture probe, electrophoresed to elongate the reporter probes, and immobilized. After processing, the sample cartridge is transferred to a fully automated imaging and data collection device (Digital Analyzer, NanoString Technologies). The level of a target is measured by imaging each sample and counting the number of times the code for that target is detected. For each sample, typically 600 fields-of-view (FOV) are imaged (1376×1024 pixels) representing approximately 10 mm2 of the binding surface. Typical imaging density is 100-1200 counted reporters per field of view depending on the degree of multiplexing, the amount of sample input, and overall target abundance. Data is output in simple spreadsheet format listing the number of counts per target, per sample. This system can be used along with nanoreporters. Additional disclosure regarding nanoreporters can be found in International Publication No. WO 07/076129 and WO07/076132, and US Patent Publication No. 2010/0015607 and 2010/0261026, the contents of which are incorporated herein in their entireties. Further, the term nucleic acid probes and nanoreporters can include the rationally designed (e.g. synthetic sequences) described in International Publication No. WO 2010/019826 and US Patent Publication No. 2010/0047924, incorporated herein by reference in its entirety.

Expression level of a gene may be expressed as absolute level or normalized level. Typically, levels are normalized by correcting the absolute level of a gene by comparing its expression to the expression of a gene that is not a relevant for determining the cancer stage of the subject, e.g., a housekeeping gene that is constitutively expressed. Suitable genes for normalization include housekeeping genes such as the actin gene ACTB, ribosomal 18S gene, GUSB, PGK1 and TFRC. This normalization allows the comparison of the level in one sample, e.g., a subject sample, to another sample, or between samples from different sources.

In particular, inventors have demonstrated that GLI1⁻/IFT20⁺ signatures, optionally GLI1⁻/IFT20⁺/PDGFRA⁻/PRKCA^{±}/FZD1⁻ signatures, GLI1⁺/IFT20⁻ signatures, optionally GLI1⁺/IFT20⁻/PDGFRA⁻/PRKCA^{±}/FZD1^{±} signatures, and GLI1⁻/IFT20⁻ signatures, optionally GLI1⁻/IFT20⁻/PDGFRA⁻/PRKCA^{±}/FZD1⁻ signatures, were linked to the presence of VDAC1-ΔC and the absence of primary cilium. In these cases and on the contrary to patients with GLI1⁺/IFT20⁺ signatures, the previously cited patients usually respond well to current treatments such as chimiotherapy and radiotherapy.

As used herein, “current treatments” is a well-known expression used by a person skilled in the art and refers to treatments administrated in first-line treatment to a patient suffering from a cancer. Current treatments usually are combinations of treatments, such as surgery with chemotherapy and/or radiotherapy. Immunotherapy, targeted therapy, or homrmotherapy may also be used following the current treatment (https://www.cancer.gov/about-cancer/treatment; https://www.who.int/cancer/treatment/en/).

As used herein, the “voltage-dependent anion channel 1 (VDAC1)” refers to a beta barrel protein that in humans is encoded by the VDAC1 gene located on chromosome 5. VDAC1 forms an ion channel in the outer mitochondrial membrane and also the outer cell membrane.

As used herein, the “VDAC1-ΔC” refers to the cleaved form of VDAC1.

In one embodiment, the detection of VDAC1-ΔC is link to the percentage of ciliated cells, in particular VDAC1-ΔC controls resorption of the primary cilium. The detection of VDAC1-ΔC is correlated with the absence of the primary cilium.

In a particular embodiment, the VDAC1-ΔC level is assessed by analyzing the expression of the protein translated from said gene. Said analysis can be assessed using an antibody (e.g., a radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from the gene encoding for the biomarkers.

Said analysis can be assessed by a variety of techniques well known from one of skill in the art including, but not limited to, enzyme immunoassay (EIA), radioimmunoassay (MA), Western blot analysis and enzyme linked immunoabsorbant assay (RIA).

In a particular embodiment, primary cilium is detected by immunofluorescence.

The method of the present invention is also suitable for determining whether a subject suffering from a cancer is eligible to anti-cancer treatment. Typically, the treatment includes chemotherapy, radiotherapy, and immunotherapy. In particular, subject having a short survival time would advantageously receive an anti-cancer treatment.

The invention relates to a method for treating a subject suffering from a cancer, wherein said method comprises the step of:
(i) Determining in a biological sample obtained from said subject the expression level of GLI1 and IFT20
(ii) Comparing the expression levels quantified at step i) with their predetermined references values and
(iii) And
   a. Administering to said subject a therapeutically effective amount of at least one therapeutic target linked to metabolism and/or immunotherapy, preferably an immune checkpoint inhibitor or an inhibitor of lactate dehydrogenase (LDH) or an inhibitor of glycolysis, if the expression level of GLI1 is higher than its predetermined reference value 1 (PRV1) and if the expression the expression level of IFT20 is higher than its predetermined references values 2 (PRV2),
   Or,
   b. Administering to said subject a therapeutically effective amount of a current treatment, preferably a tyrosine kinase inhibitor, if the expression level of GLI1 is lower than its PRV1 and if the expression level of IFT20 is higher than its PRV2
   Or,
   c. Administering to said subject a therapeutically effective amount of a current treatment, preferably a tyrosine kinase inhibitor, if the expression level of GLI1 is higher than its PRV1 and if the expression the expression level of IFT20 is lower than its PRV2
   Or,
   d. Administering to said subject a therapeutically effective amount of a current treatment, preferably a tyrosine kinase inhibitor, if the expression level of GLI1 is lower than its PRV1 and if the expression the expression level of IFT20 is lower than PRV2.

Particularly, the step ((iii) a.) consists of administering to said subject a therapeutically effective amount of an immune checkpoint inhibitor in combination with an inhibitor of lactate dehydrogenase (LDH) or an inhibitor of glycolysis if the expression level of GLI1 is higher than its predetermined reference value 1 (PRV1) and if the expression level of IFT20 is higher than its predetermined reference value 2 (PRV2).

The invention optionally relates to a method for treating a cancer treatment in a subject suffering from a cancer, wherein said method comprises the step of:
(i) Determining in a biological sample obtained from said subject the expression level of GLI family zinc finger 1 (GLI1), intraflagellar transport 20 (IFT20), platelet derived growth factor receptor alpha (PDGFRA), protein kinase C alpha (PRKCA) and frizzled class receptor 1 (FZD1)
(ii) Comparing the expression levels quantified at step i) with their predetermined references values and
(iii) And
   a) Administering to said subject a therapeutically effective amount of at least one therapeutic target linked to metabolism and/or immunotherapy, preferably an immune checkpoint inhibitor or an inhibitor of lactate dehydrogenase (LDH) or an inhibitor of glycolysis, if the expression level of GLI1 is higher than its predetermined reference value 1 (PRV1), if the expression level of IFT20 is higher than its predetermined reference value 2 (PRV2), if the expression level of PDGFRA is higher than its predetermined reference value 3 (PRV3), if the expression level of PRKCA is higher than its predetermined reference value 4 (PRV4) and if the expression level of FZD1 is lower than its predetermined reference value 5 (PRV5) or
   b) Administering to said subject a therapeutically effective amount of at least one therapeutic target linked to metabolism and/or immunotherapy, preferably an immune checkpoint inhibitor or an inhibitor of lactate dehydrogenase or an inhibitor of glycolysis, if the expression level of GLI1 is higher than its PRV1, if the expression level of IFT20 is higher than its PRV2, if the expression level of PDGFRA is lower than its PRV3, if the expression level of PRKCA is lower than its PRV4 and if the expression level of FZD1 is higher than its PRV5, or
   c) Administering to said subject a therapeutically effective amount of a current treatment, preferably a tyrosine kinase inhibitor, if the expression level of GLI1 is lower than its PRV1, if the expression level of IFT20 is higher than its PRV2, if the expression level of PDGFRA is lower than its PRV3, if the expression level of PRKCA is lower or higher than its PRV4 and if the expression level of FZD1 is lower than its PRV5 or
   d) Administering to said subject a therapeutically effective amount of a current treatment, preferably a tyrosine kinase inhibitor, if the expression level of GLI1 is higher than its PRV1, if the expression level of IFT20 is lower than its PRV2, if the expression level of PDGFRA is lower than its PRV3, if the expression level of PRKCA is lower or higher than its PRV4 and if the expression level of FZD1 is lower or higher than its PRV5 or
   e) Administering to said subject a therapeutically effective amount of a current treatment, preferably a tyrosine kinase inhibitor, if the expression level of GLI1 is lower than its PRV1, if the expression level of IFT20 is lower than its PRV2, if the expression level of PDGFRA is lower than its PRV3, if the expression level of PRKCA is lower or higher than its PRV4 and if the expression level of FZD1 is lower than its PRV5.

Particularly, the step ((iii) a.) consists of administering to the said subject a therapeutically effective amount of an immune checkpoint inhibitor in combination with an inhibitor of lactate dehydrogenase (LDH) or an inhibitor of glycolysis if the expression level of GLI1 is higher than its predetermined reference value 1 (PRV1), if the expression level of IFT20 is higher than its predetermined reference value 2 (PRV2), if the expression level of PDGFRA is higher than its predetermined reference value 3 (PRV3), if the expression level of PRKCA is higher than its predetermined reference value 4 (PRV4) and if the expression level of FZD1 is lower than its predetermined reference value 5 (PRV5) or if the expression level of GLI1 is higher than its PRV1, if the expression level of IFT20 is higher than its PRV2, if the expression level of PDGFRA is lower than its PRV3, if the expression level of PRKCA is lower than its PRV4 and if the expression level of FZD1 is higher than its PRV5.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

In some embodiments, the treatment consists of administering to the subject a targeted cancer therapy. Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs,” “molecularly targeted therapies,” “precision medicines,” or similar names.

In some embodiments, the treatment consists of administering to the subject an immunotherapeutic agent. The term “immunotherapeutic agent,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively the immunotherapeutic treatment may consist of administering the subject with an amount of immune cells (T cells, NK, cells, dendritic cells, B cells . . . ).

Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents.

Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors.

Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation).

Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention.

Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin).

In addition to having specific or non-specific targets, immunotherapeutic agents can be active, i.e. stimulate the body's own immune response, or they can be passive, i.e. comprise immune system components that were generated external to the body.

Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins.

In some embodiments, the immunotherapeutic agent is an immune checkpoint inhibitor.

In some embodiments, the targeted therapy consists of administering the subject with an immune checkpoint inhibitor.

As used herein, the term “immune checkpoint inhibitor” refers to molecules that totally or partially reduce, inhibit, interfere with or modulate one or more immune checkpoint proteins. As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al. 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein. Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e.g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to —N-(3-bromo-4-fluorophenyl)-N′-hydroxy-4-{[2-(sulfamoylamino)-éthyl]amino}-1,2,5-oxadiazole-3 carboximidamide:

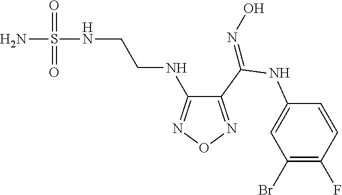 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

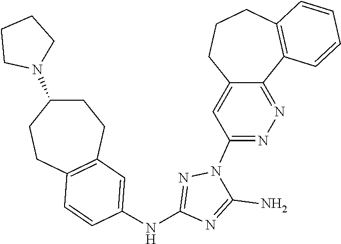 [SMILES: Nc1nc(Nc2ccc3c(c2)CC[C@@H](N2CCCC2)CC3)nn1-c1cc2c(nn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

In some embodiments, the targeted therapy consists of administering the subject with a lactate dehydrogenase inhibitor.

As used herein, the term “lactate deshydrogenase” (LDH) refers to a tetrameric enzyme comprising two major subunits A and/or B, resulting in five isozymes (A4, A3B1, A2B2, A1B3, and B4) that can catalyze the forward and backward conversion of pyruvate to lactate. LDHA (LDH-5, MLDH, or A4), which is the predominant form in skeletal muscle, kinetically favors the conversion of pyruvate to lactate. LDHB (LDH-1, H-LDH, or B4), which is found in heart muscle, converts lactate to pyruvate that is further oxidized.

As used herein, the term “inhibitor of lactate dehydrogenase” refers to inhibitors of the lactate deshydrogenase. Examples of lactate dehydrogenase inhibitors and related compounds suitable for use in methods of embodiments of the present invention include, but are not limited to dichloroacetate (DCA), FX11, AZD-3965, Oxamate (OXA), Galloflavin (GF), Gossypol, Quinoline 3-sulfonamides, N-hydroxyindole-based (NHI) inhibitors, bifunctional ligands, Mn(II) complexes.

As used herein, the term “dichloroacetate” also known as “DCA” has the formula C₂H₂Cl₂O₂ and the following structure in the art:

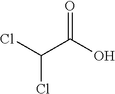

As used herein, the term “FX11” has the formula C₂₂H₂₂O₄ C₂₁H₂₄F₃N₅O₅S and the following structure in the art:

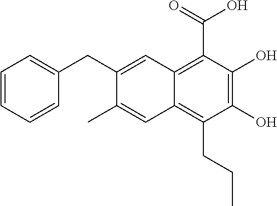 [SMILES: CCCc1c(O)c(O)c(C(=O)O)c2cc(Cc3ccccc3)c(C)cc12]

As used herein, the term “AZD-3965” also called 5-[[(4S)-4-hydroxy-4-methyl-2-isoxazolidinyl]carbonyl]-3-methyl-1-(1-methylethyl)-6-[[5-methyl-3-(trifluoromethyl)-1H-pyrazol-4-yl]methyl]-thieno[2,3-d]pyrimidine-2,4(1H,3H)-dione has the formula C₂₁H₂₄F₃N₅O₅S.

In some embodiments, the targeted therapy consists of administering the subject with a glycolysis inhibitor.

As used herein, the term “inhibitor of glycolysis” refers to inhibitors of the glycolysis. Examples of glycolysis inhibitors and related compounds suitable for use in methods of embodiments of the present invention include, but are not limited to 3-bromopyruvate, 2-Deoxyglucose, Lonidamine, Imatinib, Oxythiamine.

As used herein, the term “3-bromopyruvate” also known as “3BP” has the formula BrCH₂COCO₂H and the following structure in the art:

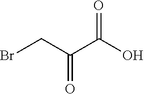

In some embodiments, the targeted therapy consists of administering to the subject a lactate dehydrogenase inhibitor or glycolysis inhibitor.

In some embodiments, the targeted therapy consists of administering the subject with a tyrosine kinase inhibitor.

As used herein, the term “tyrosine kinase inhibitor” refers to any of a variety of therapeutic agents or drugs that act as selective or non-selective inhibitors of receptor and/or non-receptor tyrosine kinases. Tyrosine kinase inhibitors and related compounds are well known in the art and described in U.S Patent Publication 2007/0254295, which is incorporated by reference herein in its entirety. It will be appreciated by one of skill in the art that a compound related to a tyrosine kinase inhibitor will recapitulate the effect of the tyrosine kinase inhibitor, e.g., the related compound will act on a different member of the tyrosine kinase signaling pathway to produce the same effect as would a tyrosine kinase inhibitor of that tyrosine kinase. Examples of tyrosine kinase inhibitors and related compounds suitable for use in methods of embodiments of the present invention include, but are not limited to, sunitinib (Sutent; SU11248), dasatinib (BMS-354825), PP2, BEZ235, saracatinib, gefitinib (Iressa), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; STI571), leflunomide (SU101), vandetanib (Zactima; ZD6474), MK-2206 (8-[4-aminocyclobutyl)phenyl]-9-phenyl-1,2,4-triazolo[3,4-f][1,6]naphthyridin-3(2H)-one hydrochloride) derivatives thereof, analogs thereof, and combinations thereof. Additional tyrosine kinase inhibitors and related compounds suitable for use in the present invention are described in, for example, U.S Patent Publication 2007/0254295, U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340, all of which are incorporated by reference herein in their entirety. In certain embodiments, the tyrosine kinase inhibitor is a small molecule kinase inhibitor that has been orally administered and that has been the subject of at least one Phase I clinical trial, more preferably at least one Phase II clinical, even more preferably at least one Phase III clinical trial, and most preferably approved by the FDA for at least one hematological or oncological indication. Examples of such inhibitors include, but are not limited to, Gefitinib, Erlotinib, Lapatinib, Canertinib, BMS-599626 (AC-480), Neratinib, KR-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KR-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901.

In a particular embodiment, the tyrosine kinase inhibitor is sunitinib.

As used herein, the term “sunitinib” also called “N-[2-(diethylamino)ethyl]-5-{[(3Z)-5-fluoro-2-oxo-2,3-dihydro-1H-indol-3-ylidene]methyl}-2,4-dimethyl-1H-pyrrole-3-carboxamide” has its general meaning in the art and refers to the compound characterized by the formula of:

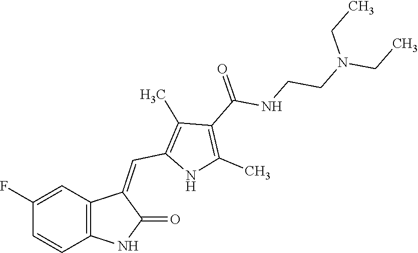 [SMILES: CCN(CC)CCNC(=O)c1c(C)[nH]c(/C=C2\C(=O)Nc3ccc(F)cc32)c1C]

In some embodiments, the subject will be treated only with an inhibitor of lactate dehydrogenase (LDH) if the expression level of GLI1 is higher than its predetermined reference value 1 (PRV1) and if the expression level of IFT20 is higher than its predetermined reference value 2 (PRV2).

In some embodiments, the subject will be treated only with an inhibitor of lactate dehydrogenase (LDH) if the expression level of GLI1⁺ is detected and if the expression level of IFT20⁺ is detected.

In some embodiments, the subject will be treated only with an inhibitor of lactate dehydrogenase (LDH) if the expression level of GLI1 is higher than its predetermined reference value 1 (PRV1), if the expression level of IFT20 is higher than its predetermined reference value 2 (PRV2), if the expression level of PDGFRA is higher than its predetermined reference value 3 (PRV3), if the expression level of PRKCA is higher than its predetermined reference value 4 (PRV4 and if the expression level of FZD1 is lower than its predetermined reference value 5 (PRV5).

In some embodiments, the subject will be treated only with an inhibitor of lactate dehydrogenase (LDH) if the expression level of GLI1⁺ is detected, if the expression level of IFT20⁺ is detected, if the expression level of PDGFRA is detected, if the expression level of PRKCA⁺ is detected and if the expression level of FZD1⁻ is detected.

In some embodiments, the subject will be treated only with an inhibitor of lactate dehydrogenase (LDH) if the expression level of GLI1 is higher than its predetermined reference value 1 (PRV1), if the expression level of IFT20 is higher than its predetermined reference value 2 (PRV2), if the expression level of PDGFRA is lower than its predetermined reference value 3 (PRV3), if the expression level of PRKCA is lower than its predetermined reference value 4 (PRV4 and if the expression level of FZD1 is higher than its predetermined reference value 5 (PRV5).

In some embodiments, the subject will be treated only with an inhibitor of lactate dehydrogenase (LDH) if the expression level of GLI1⁺ is detected, if the expression level of IFT20⁺ is detected, if the expression level of PDGFRA⁻ is detected, if the expression level of PRKCA⁻ is detected and if the expression level of FZD1⁺ is detected.

In some embodiments, the subject will be treated only with an inhibitor of glycolysis if the expression level of GLI1 is higher than its predetermined reference value 1 (PRV1) and if the expression level of IFT20 is higher than its predetermined reference value 2 (PRV2).

In some embodiments, the subject will be treated only with an inhibitor of glycolysis if the expression level of GLI1⁺ is detected and if the expression level of IFT20⁺ is detected.

In some embodiments, the subject will be treated only with an inhibitor of glycolysis if the expression level of GLI1 is higher than its predetermined reference value 1 (PRV1), if the expression level of IFT20 is higher than its predetermined reference value 2 (PRV2), if the expression level of PDGFRA is higher than its predetermined reference value 3 (PRV3), if the expression level of PRKCA is higher than its predetermined reference value 4 (PRV4 and if the expression level of FZD1 is lower than its predetermined reference value 5 (PRV5).

In some embodiments, the subject will be treated only with an inhibitor of glycolysis if the expression level of GLI1⁺ is detected, if the expression level of IFT20⁺ is detected, if the expression level of PDGFRA is detected, if the expression level of PRKCA⁺ is detected and if the expression level of FZD1⁻ is detected.

In some embodiments, the subject will be treated only with an inhibitor of glycolysis if the expression level of GLI1 is higher than its predetermined reference value 1 (PRV1), if the expression level of IFT20 is higher than its predetermined reference value 2 (PRV2), if the expression level of PDGFRA is lower than its predetermined reference value 3 (PRV3), if the expression level of PRKCA is lower than its predetermined reference value 4 (PRV4 and if the expression level of FZD1 is higher than its predetermined reference value 5 (PRV5).

In some embodiments, the subject will be treated only with an inhibitor of glycolysis if the expression level of GLI1⁺ is detected, if the expression level of IFT20⁺ is detected, if the expression level of PDGFRA⁻ is detected, if the expression level of PRKCA⁻ is detected and if the expression level of FZD1⁺ is detected.

In some embodiments, the subject will be treated only with an immune checkpoint inhibitor if the expression level of GLI1 is higher than its predetermined reference value 1 (PRV1) and if the expression level of IFT20 is higher than its predetermined reference value 2 (PRV2).

In some embodiments, the subject will be treated only with an immune checkpoint inhibitor if the expression level of GLI1⁺ is detected and if the expression level of IFT20⁺ is detected.

In some embodiments, the subject will be treated only with an immune checkpoint inhibitor if the expression level of GLI1 is higher than its predetermined reference value 1 (PRV1), if the expression level of IFT20 is higher than its predetermined reference value 2 (PRV2), if the expression level of PDGFRA is higher than its predetermined reference value 3 (PRV3), if the expression level of PRKCA is higher than its predetermined reference value 4 (PRV4 and if the expression level of FZD1 is lower than its predetermined reference value 5 (PRV5).

In some embodiments, the subject will be treated only with an immune checkpoint inhibitor if the expression level of GLI1⁺ is detected, if the expression level of IFT20⁺ is detected, if the expression level of PDGFRA⁺ is detected, if the expression level of PRKCA is detected and if the expression level of FZD1⁻ is detected.

In some embodiments, the subject will be treated only with an immune checkpoint inhibitor if the expression level of GLI1 is higher than its predetermined reference value 1 (PRV1), if the expression level of IFT20 is higher than its predetermined reference value 2 (PRV2), if the expression level of PDGFRA is lower than its predetermined reference value 3 (PRV3), if the expression level of PRKCA is lower than its predetermined reference value 4 (PRV4 and if the expression level of FZD1 is higher than its predetermined reference value 5 (PRV5).

In some embodiments, the subject will be treated only with an immune checkpoint inhibitor if the expression level of GLI1⁺ is detected, if the expression level of IFT20⁺ is detected, if the expression level of PDGFRA⁻ is detected, if the expression level of PRKCA⁻ is detected and if the expression level of FZD1⁺ is detected.

In some embodiments, the targeted therapy consists of administering to the subject an immune checkpoint inhibitor in combination with a lactate dehydrogenase inhibitor.

In some embodiments, the targeted therapy consists of administering to the subject an immune checkpoint inhibitor in combination with a glycolysis inhibitor.

In a particular embodiment, i) an immune checkpoint inhibitor and ii) a lactate dehydrogenase inhibitor as a combined preparation according to the invention for simultaneous, separate or sequential use in the method for treating a cancer in a subject.

In a particular embodiment, i) an immune checkpoint inhibitor and ii) a glycolysis inhibitor as a combined preparation according to the invention for simultaneous, separate or sequential use in the method for treating a cancer in a subject.

As used herein, the term “combination” is intended to refer to all forms of administration that provide a first drug together with a further (second, third . . . ) drug. The drugs may be administered simultaneous, separate or sequential and in any order. According to the invention, the drug is administered to the subject using any suitable method that enables the drug to reach the lungs. In some embodiments, the drug administered to the subject systemically (i.e. via systemic administration). Thus, in some embodiments, the drug is administered to the subject such that it enters the circulatory system and is distributed throughout the body. In some embodiments, the drug is administered to the subject by local administration, for example by local administration to the lungs.

As used herein, the terms “combined treatment”, “combined therapy” or “therapy combination” refer to a treatment that uses more than one medication. The combined therapy may be dual therapy or bi-therapy.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

In some embodiments, the subject will be treated with an immune checkpoint inhibitor in combination with an inhibitor of lactate dehydrogenase (LDH) if the expression level of GLI1 is higher than its predetermined reference value 1 (PRV1) and if the expression level of IFT20 is higher than its predetermined reference value 2 (PRV2).

In some embodiments, the subject will be treated with an immune checkpoint inhibitor in combination with an inhibitor of lactate dehydrogenase (LDH) if the expression level of GLI1⁺ is detected and if the expression level of IFT20⁺ is detected.

In some embodiments, the subject will be treated with an immune checkpoint inhibitor in combination with an inhibitor of lactate dehydrogenase (LDH) if the expression level of GLI1 is higher than its predetermined reference value 1 (PRV1), if the expression level of IFT20 is higher than its predetermined reference value 2 (PRV2), if the expression level of PDGFRA is higher than its predetermined reference value 3 (PRV3), if the expression level of PRKCA is higher than its predetermined reference value 4 (PRV4 and if the expression level of FZD1 is lower than its predetermined reference value 5 (PRV5).

In some embodiments, the subject will be treated with an immune checkpoint inhibitor in combination with an inhibitor of lactate dehydrogenase (LDH) if the expression level of GLI1⁺ is detected, if the expression level of IFT20⁺ is detected, if the expression level of PDGFRA is detected, if the expression level of PRKCA is detected and if the expression level of FZD1⁻ is detected.

In some embodiments, the subject will be treated with an immune checkpoint inhibitor in combination with an inhibitor of lactate dehydrogenase (LDH) if the expression level of GLI1 is higher than its predetermined reference value 1 (PRV1), if the expression level of IFT20 is higher than its predetermined reference value 2 (PRV2), if the expression level of PDGFRA is lower than its predetermined reference value 3 (PRV3), if the expression level of PRKCA is lower than its predetermined reference value 4 (PRV4 and if the expression level of FZD1 is higher than its predetermined reference value 5 (PRV5).

In some embodiments, the subject will be treated with an immune checkpoint inhibitor in combination with an inhibitor of lactate dehydrogenase (LDH) if the expression level of GLI1⁺ is detected, if the expression level of IFT20⁺ is detected, if the expression level of PDGFRA⁻ is detected, if the expression level of PRKCA⁻ is detected and if the expression level of FZD1⁺ is detected.

In some embodiments, the subject will be treated with an immune checkpoint inhibitor in combination with an inhibitor of glycolysis if the expression level of GLI1 is higher than its predetermined reference value 1 (PRV1) and if the expression level of IFT20 is higher than its predetermined reference value 2 (PRV2).

In some embodiments, the subject will be treated with an immune checkpoint inhibitor in combination with an inhibitor of glycolysis if the expression level of GLI1⁺ is detected and if the expression level of IFT20⁺ is detected.

In some embodiments, the subject will be treated with an immune checkpoint inhibitor in combination with an inhibitor of glycolysis if the expression level of GLI1 is higher than its predetermined reference value 1 (PRV1), if the expression level of IFT20 is higher than its predetermined reference value 2 (PRV2), if the expression level of PDGFRA is higher than its predetermined reference value 3 (PRV3), if the expression level of PRKCA is higher than its predetermined reference value 4 (PRV4 and if the expression level of FZD1 is lower than its predetermined reference value 5 (PRV5).

In some embodiments, the subject will be treated with an immune checkpoint inhibitor in combination with an inhibitor of glycolysis if the expression level of GLI1⁺ is detected, if the expression level of IFT20⁺ is detected, if the expression level of PDGFRA⁺ is detected, if the expression level of PRKCA⁺ is detected and if the expression level of FZD1⁻ is detected.

In some embodiments, the subject will be treated with an immune checkpoint inhibitor in combination with an inhibitor of glycolysis if the expression level of GLI1 is higher than its predetermined reference value 1 (PRV1), if the expression level of IFT20 is higher than its predetermined reference value 2 (PRV2), if the expression level of PDGFRA is lower than its predetermined reference value 3 (PRV3), if the expression level of PRKCA is lower than its predetermined reference value 4 (PRV4 and if the expression level of FZD1 is higher than its predetermined reference value 5 (PRV5).

In some embodiments, the subject will be treated with an immune checkpoint inhibitor in combination with an inhibitor of glycolysis if the expression level of GLI1⁺ is detected, if the expression level of IFT20⁺ is detected, if the expression level of PDGFRA⁻ is detected, if the expression level of PRKCA⁻ is detected and if the expression level of FZD1⁺ is detected.

In some embodiments, the subject will be treated with a tyrosine kinase inhibitor if the expression level of GLI1 is higher than its predetermined reference value 1 (PRV1) and if the expression level of IFT20 is lower than its predetermined reference value 2 (PRV2).

In some embodiments, the subject will be treated with a tyrosine kinase inhibitor if the expression level of GLI1⁺ is detected and if the expression level of IFT20⁻ is detected.

In some embodiments, the subject will be treated with a tyrosine kinase inhibitor if the expression level of GLI1 is higher than its predetermined reference value 1 (PRV1), if the expression level of IFT20 is lower than its predetermined reference value 2 (PRV2), if the expression level of PDGFRA is lower than its predetermined reference value 3 (PRV3), if the expression level of PRKCA is lower or higher than its predetermined reference value 4 (PRV4 and if the expression level of FZD1 is lower or higher than its predetermined reference value 5 (PRV5).

In some embodiments, the subject will be treated with a tyrosine kinase inhibitor if the expression level of GLI1⁺ is detected, if the expression level of IFT20⁻ is detected, if the expression level of PDGFRA⁻ is detected, if the expression level of PRKCA^{±} is detected and if the expression level of FZD1^{±} is detected.

In some embodiments, the subject will be treated with a tyrosine kinase inhibitor if the expression level of GLI1 is lower than its predetermined reference value 1 (PRV1) and if the expression level of IFT20 is lower than its predetermined reference value 2 (PRV2).

In some embodiments, the subject will be treated with a tyrosine kinase inhibitor if the expression level of GLI1⁻ is detected and if the expression level of IFT20⁻ is detected.

In some embodiments, the subject will be treated with a tyrosine kinase inhibitor if the expression level of GLI1 is lower than its predetermined reference value 1 (PRV1), if the expression level of IFT20 is lower than its predetermined reference value 2 (PRV2), if the expression level of PDGFRA is lower than its predetermined reference value 3 (PRV3), if the expression level of PRKCA is lower or higher than its predetermined reference value 4 (PRV4 and if the expression level of FZD1 is lower than its predetermined reference value 5 (PRV5).

In some embodiments, the subject will be treated with a tyrosine kinase inhibitor if the expression level of GLI1⁻ is detected, if the expression level of IFT20⁻ is detected, if the expression level of PDGFRA⁻ is detected, if the expression level of PRKCA^{±} is detected and if the expression level of FZD1⁻ is detected.

In some embodiments, the subject will be treated with a tyrosine kinase inhibitor if the expression level of GLI1 is lower than its predetermined reference value 1 (PRV1) and if the expression level of IFT20 is higher than its predetermined reference value 2 (PRV2).

In some embodiments, the subject will be treated with a tyrosine kinase inhibitor if the expression level of GLI1⁻ is detected and if the expression level of IFT20+ is detected.

In some embodiments, the subject will be treated with a tyrosine kinase inhibitor if the expression level of GLI1 is lower than its predetermined reference value 1 (PRV1), if the expression level of IFT20 is higher than its predetermined reference value 2 (PRV2), if the expression level of PDGFRA is lower than its predetermined reference value 3 (PRV3), if the expression level of PRKCA is lower or higher than its predetermined reference value 4 (PRV4 and if the expression level of FZD1 is lower than its predetermined reference value 5 (PRV5).

In some embodiments, the subject will be treated with a tyrosine kinase inhibitor if the expression level of GLI1⁻ is detected, if the expression level of IFT20⁺ is detected, if the expression level of PDGFRA⁻ is detected, if the expression level of PRKCA^{±} is detected and if the expression level of FZD1⁻ is detected.

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agent of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Typically, the immune checkpoint inhibitor or the lactate dehydrogenase inhibitor or the glycolysis inhibitor or the tyrosine kinase inhibitor as described above are administered to the subject in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a subject, the composition will be formulated for administration to the subject. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

The invention will be further illustrated by the following figures and examples. However, these examples and figures should not be interpreted in any way as limiting the scope of the present invention.

### FIGURES

FIG. 1: Schematic representation of the impact of VDAC1-ΔC on biogenesis of the primary cilium. The presence of VDAC1-ΔC and the GLI1/IFT20/PDGFRA/PRKCA/FZD1 signature are new markers for classification of ccRCC patient. Tumors from ccRCC patients that express primary cilia (12-18%) with a GLI1+/IFT20+ signature but not VDAC1-ΔC (−) (groups PC+) are significantly more aggressive with a bad prognosis than groups 0/1 and 2 that express low primary cilia (0.7-3.7%). Therapeutic approaches are proposed: tyrosine kinase inhibitor treatment for groups PC−, and inhibitor of lactate dehydrogenase or inhibitor of glycolysis±immunotherapy for groups PC+.

FIG. 2. Identification and validation of the 5-gene signature predictive of the presence of primary cilia and of the aggressiveness of tumors of ccRCC patients from Cohort B and from TCGA (Cohort C). (A) Disease free survival for the primary cilium signature was calculated from patients of the cohort C using the GLI1/IFT20 signature. Patients without a primary cilium (PC−) were GLI1−/IFT20−, GLI1+/IFT20− or GLI1−/IFT20+. Patients with primary cilia (PC+) were GLI1+/IFT20+. Statistical significance (p-value) is indicated. The median survival is also indicated. (B) Overall survival for the primary cilium signature was calculated from patients of the TCGA cohort C using the GLI1/IFT20 signature. Patients without a primary cilium (PC−) were GLI1−/IFT20−, GLI1+/IFT20− or GLI1−/IFT20+. Patients with primary cilia (PC+) were GLI1+/IFT20+. Statistical significance (p-value) is indicated. The median survival is also indicated.

FIG. 3. Patients with primary cilium signature present a higher immunogenicity compared to no primary cilium signature and a better response to immunotherapy. (A) Progression free-survival (PFS) and (B) Overall survival (OS) for the primary cilium signature was calculated from patients of the TCGA cohort C treated with sunitinib using the GLI1/IFT20 signature. Statistical significance (p-value) is indicated. The median survival is also indicated.

FIG. 4: Patients with primary cilium signature present a higher immunogenicity compared to no primary cilium signature and a better response to immunotherapy. (A) Tumors from patients with no primary signature and tumors from patients with primary cilium were compared. The level of PD1 mRNA was determined by qPCR for the TCGA cohort. Statistical significance (p value) is indicated. (B) Distribution of ccRCC patients from the TCGA database depending on the primary cilium (PC) signature and immunophenoscore. p-value between no primary cilium and primary cilium is indicated.

FIG. 5: (A) Graphic representation of PDL1 mRNA expression in patients from Group A compared to patients from Group B. (B) Representative image of immunofluorescence analysis of patient #8 (Group A) and patient #13 (Group B) studied to evaluate a prediction model of the absence or presence of PDL1 expression. A * p<0.05 show significant differences. PDL1 is present in group B.

### EXAMPLE

Material & Methods

Cell Culture

RCC4/pVHL and 786-O/pVHL cells were grown in Dulbecco's Modified Eagle's Medium (DMEM) (Gibco-BRL) supplemented with 10% fetal bovine serum with penicillin G (50 U/ml) and streptomycin sulfate (50 μg/ml). ACHN and A498 RCC cell lines were purchased from the ATCC (Mar. 3, 2013). The RCC10 cell line was a kind gift from Dr. W. H. Kaelin (Dana-Farber Cancer Institute, Boston, Mass.). An INVIVO2 200 anaerobic workstation (Ruskinn Technology Biotrace International Plc) set at 1% oxygen, 94% nitrogen and 5% carbon dioxide was used for hypoxic conditions.

Pharmacological Inhibitors and Chemicals

Rotenone, antimycin A, oligomycin, trifluorocarbonylcyanide phenylhydrazone (FCCP) and 3BP were from Sigma. Sunitinib was from Centre Antoine Lacassagne.

RNA Interference

The 21-nucleotide RNAs were chemically synthesized (Eurogentec, Seraing, Belgium). The siRNA sequences were as follows: siCtl (forward) 5′-CCU-ACA-UCC-CGA-UCG-AUG-AUG-TT-3′(SEQ ID NO: 1), siVDAC1 (forward) 5′-GAUACACUCAGACUCUAAA-3′ (SEQ ID NO: 2), siHIF-1α (forward) 5′-CUG-AUG-ACCAGC-AAC-UUG-ATT-3′(SEQ ID NO: 3), siHIF-2a (forward) 5′-CAG-CAU—CUU-UGA-UAG-CAG-UTT-3′(SEQ ID NO: 4). siIFT20 and siGLI1 were from Mission esiRNA Sigma. siAEP was from EUROGENTECH.

Colony-Forming Assay

Cells (5000-10000) were plated on 60 mm dishes and incubated at 37° C., 5% CO2 for colony formation. After 10 days, colonies were fixed with 10% (v/v) methanol for 15 min and stained with 5% Giemsa (Sigma) for 30 min for colony visualization.

Respirometry and Extracellular Acidification

The cellular oxygen consumption rate (OCR) and extracellular acidification rate (ECAR) were obtained using a Seahorse XF24 extracellular flux analyzer from Seahorse Bioscience (North Billerica, Mass., USA). Experiments were performed according to the manufacturer's instructions. The Oxygen Consumption Rate (OCR) and the ExtraCellular Acidification Rate (ECAR) were measured in real time in normoxia or hypoxia. Cells were deprived of glucose for 1 h, then glucose (G—10 mM), oligomycin (O—104), FCCP (F—304) and Rotenone+Antimycin A (R/A—1 μM) were injected at the indicated times. Protein standardization was performed after each experiment, with no noticeable differences in protein concentration and cell phenotype.

Quantitative Real-Time PCR Analysis

For tumor samples, total RNA was extracted with RNeasy FFPE Kit (QIAGEN, Hilden, Germany). For cells, total RNA was extracted with the RNeasy Mini Kit (QIAGEN, Hilden, Germany). The amount of RNA was evaluated with a NanoDrop™ spectrophotometer (ThermoFisher Scientific, Waltham, Mass. USA). One μg of total RNA was used for reverse transcription, using the QuantiTect Reverse Transcription kit (QIAGEN, Hilden, Germany), with a blend of oligo (dT) and random primers to prime first-strand synthesis. SYBR master mix plus (Eurogentec, Liege, Belgium) was used for qPCR and specific oligonucleotides (Sigma Aldrich). Primer sequences used are: GLI1 (forward: 5′-TGCAGTAAAGCCTTCAGCAATG-3′ (SEQ ID NO: 5); reverse: 5′-TTTTCGCAGCGAGCTAGGAT-3′ (SEQ ID NO: 6)), IFT20 (forward: 5′-GGTATCGGGTTGAATATGAAG-3′ (SEQ ID NO: 7); reverse: 5′-GACATAGGTCATTGGTCAAG-3′ (SEQ ID NO: 8)), PDGFRA (forward: 5′-TCAAGTTCCTTCATCCATTC-3′ (SEQ ID NO: 9); reverse: 5′-CATCCACTCAATATCAGGAAG-3′ (SEQ ID NO: 10)), PRKCA (forward: 5′-CCAAAGTGTGTGGCAAAG-3′ (SEQ ID NO: 11); reverse: 5′-TCAGACTGGTCTATGTTAGC-3′ (SEQ ID NO: 12)) and FZD1 (forward: 5′-ACTGCATTAAATCCTGTGTG-3′ (SEQ ID NO: 13); reverse: 5′-GCTTTTCTCCTCTTCTTCAC-3′ (SEQ ID NO: 14)) LGMN (forward: 5′-ACTATGATGAGAAGAGGTCC-3′(SEQ ID NO: 15); reverse: 5′-GGTGGAGATTGTTTTGTTTC-3′(SEQ ID NO: 16)); PDL1 (forward: 5′-ATGCCCCATACAACAAAATC-3′(SEQ ID NO: 17); reverse: 5′-GACATGTCAGTTCATGTTCAG-3′ ((SEQ ID NO: 18)); STAT3 primers was a kind gift from Dr J. Gilleron (C3M, Nice).

Immunoblotting

Cells were lysed in 1.5×SDS buffer and the protein concentration determined using the BCA assay. 40 μg of protein from whole cell extracts was resolved by SDS-PAGE and transferred onto a PVDF membrane (Millipore). Membranes were blocked in 5% non-fat milk in TN buffer (50 mM Tris-HCl pH7.4, 150 mM NaCl) and incubated in the presence of the primary and then secondary antibodies in 5% non-fat milk in TN buffer. The rabbit polyclonal antibody to central regions of VDAC1 was purchased from Abcam (ab15895). Rabbit polyclonal anti-HIF-1α antibody (antiserum 2087) was produced and characterized in our laboratory (25). The antibodies against HIF-2a (NB100-122) and ARL13b (NBP2-15463) were purchased from Novus Biologicals (Littleton, Calif.). Anti-BNIP3 was described previously (26) and purchased from Abcam. Anti-LC3 was raised in rabbits immunized against the N-terminal 14 amino acids of human LC3 and was produced and characterized in our laboratory (26). Mouse anti-acetylated tubulin (T7451), anti-β-tubulin, anti-α-tubulin and β-actin were from Sigma. ECL signals were normalized to either β-tubulin or ARD1 (27). After washing in TN buffer containing 1% Triton-X100 and then in TN buffer, immunoreactive bands were visualized with the ECL system (Amersham Biosciences).

Immunocytochemistry

Cells were fixed in 3% paraformaldehyde and extracted with Triton X-100. Primary antibodies included mouse anti-acetylated tubulin (Sigma-Aldrich, Basel, Switzerland) (1:400); rabbit anti-Arl13b (Novusbio, Abingdon, United Kingdom) (1:400 dilution). Alexa Fluor 594- and 488-conjugated secondary goat anti-mouse or goat anti-rabbit antibodies (Molecular Probes, Carlsbad, Calif.) were used at 1:400. Cells were visualized by wide-field, fluorescence microscopy using a DM5500B upright stand (Leica, Germany) with a 40× oil objective NA 1.00. The cubes used were A4 (excitation filter BP 360/40, dichroic mirror 400, emission filter BP 470/40), L5 (BP 480/40, 505, BP 527/30), and TX2 (BP 560/40, 595, BP645/75). Acquisitions were done with an Orca-ER camera (Hamamatsu, Japan). Cells were also visualized using the confocal microscope, Axiovert 200M inverted stand (Zeiss, Germany). Objectives 10× dry NA 0.3 and/or 25× multi immersion (oil, glycerol, water) NA 0.75, and/or 40× oil 1.3 NA and/or 63× oil 1.4 NA were used. The LASER used were diode 405 nm, and/or Argon 488 nm, and/or HeNe 543 nm. The microscope was equipped with an automated xy stage for mosaic acquisitions. Cilia frequency were counted manually from scans using a 40× digital zoom for 100-300 nuclei.

Immunohistochemistry

Normal and renal cell carcinoma tissue sections (5 μm) were obtained from the Department of Pathology of Centre Hospitalier Universitaire de Nice, Nice (France). After dewaxing, rehydrating, antigen retrieval was achieved by boiling in 0.01M citrate buffer for 20 min. For immunofluorescence detection of primary cilia, sections were incubated with mouse antiacetylated tubulin (Sigma-Aldrich, Basel, Switzerland) (1:50 dilution) and rabbit anti-Ar113b (Novusbio, Abingdon, United Kingdom) (1:50 dilution) primary antibodies and goat antimouse secondary antibodies conjugated to Alexa 594 and goat anti-rabbit secondary antibodies conjugated to Alexa 488 (Molecular Probes, Invitrogen, Basel, Switzerland) (1:100 dilution). Nuclei were labeled with 2 mg/ml 4-6-diamidino-2-phenylindole (DAPI). Cilia counting was performed by focusing up and down on the microscope to capture cilia and nuclei that lay in different focal planes within the section. Images were obtained using a Axiovert 200M inverted stand (Zeiss, Germany) with a 40× oil objective 1.3 NA with samples mounted in an immersion medium (water). A diode 405 nm, Argon 488 nm and HeNe 543 nm laser was used. Optical sections were 0.3 μm thick and stacks were made encompassing a Zplane depth of 0.5 μm. The number of cilia was counted manually from scans using a 40× digital zoom for at least 500 nuclei.

Invasion Assay

The invasion assay was performed using cell culture inserts with 8.0 μm pore transparent PET membrane coated with 10 μg/mL fibronectin. Inserts were coated with 2 μg/μL of Matrigel and incubated for 3 h at 37° C. in a CO2 incubator. Briefly, overnight serum-starved cells (8×104 cells) were seeded into the top chamber in medium without FBS, while medium with 10% FBS was present in the bottom chamber. The cells were incubated for 24 h. Media and remaining cells were removed from the top chamber with a cotton swab and washed twice with PBS. The bottom chamber was aspirated and washed twice with PBS. Inserts were fixed with 4% PFA. Invasiveness was assayed in triplicate for each condition, in at least three independent experiments. Cells that invaded the Matrigel and migrated through the filter and adhered to the lower surface were stained for 10 min with 0.5% crystal violet in 25% methanol. Inserts were rinsed in distilled water until no additional stain leached and were air-dried overnight. Crystal violet was extracted from the invading cells by adding 600 μl of 0.1M sodium citrate in 10% acetic acid. Absorbance was measured spectrophotometrically at 585 nm using Spectronic GENESYS 5 (Milton Roy, Rochester, N.Y.). Microarray experiments Microarray experiments were already described (22). The experimental data have been deposited on the NCBI Gene Expression Omnibus (GEO) (http://www.ncbi.nlm.nih.gov/geo/) under the series record number GSE63247.

Patients and Cohorts

RCC is classified according to the tumor, node and metastasis (TNM) system developed by the American Joint Committee on Cancer (AJCC). RCC is staged from Stage I to Stage IV and is determined with the TN and is a pronostic score.

Stage Groupings:

stage I: T1 N0 M0

stage II: T2 N0 M0

stage III: T3 or N1 with M0

stage IV: T4 or M1

Patients from Nice (data not shown): Tissue samples from 12 patients with ccRCC who had undergone surgery in the Urology and Pathology Departments of the Nice University Hospital were selected (data not shown). For each patient, a piece of fresh tumor was embedded in paraffin (IF) and a piece was immediately frozen (immunoblot). For each patient, tumor diagnosis, as defined by the 2016 World Health Organization criteria, was based on pathology and on cytogenetic analyses. This prospective study was approved by the institutional review board and was conducted in accordance with the Declaration of Helsinki.

Cohort A (PREDIR) (data not shown): A series of 32 renal tumors were obtained thanks to the PREDIR Center (French Kidney Cancer Consortium coordinated by S. Richard). It is composed of 12 VHL tumor-associated and 22 sporadic RCC verified as being clear-cell renal cell carcinomas. Part of the transcriptome analysis by microarray of this series was previously reported (28), in press).

Cohort B (data not shown) Tissue samples from 43 patients with ccRCC who had undergone surgery in the Urology Department of the Rennes University Hospital were selected (data not shown). As defined by the 2016 World Health Organization criteria, diagnosis was based on the pathology and on cytogenetic analyses. This retrospective study was approved by the institutional review board and was conducted in accordance with the Declaration of Helsinki.

Cohort C has been previously described (29)

Gene Expression Microarray Analysis

Normalized RNA sequencing (RNA-Seq) data produced by The Cancer Genome Atlas (TCGA) were downloaded from cbioportal (www.cbioportal.org, TCGA Provisional; RNA-Seq V2). Different parameters were available for 375 ccRCC tumor samples, with information for VHL status (methylation, mutation and deletion) (30,31). We then performed a differential expression analysis between patients with a primary cilium signature and patients with a no primary cilium signature using the Bioconductor package DESeq2. The results published here are in whole or in part based upon data generated by the TCGA Research Network.

Database Analysis

To assess the effect of the presence or absence of the primary cilium in ccRCC of the Cohort C, we performed a differential analysis between the group expressing the primary cilium (n=48 patients) and the group expressing no primary cilium (n=327 patients) by computing the ratio and p-values obtained with a Wilcoxon rank sum test. We then performed a functional and pathway enrichment analysis on differentially expressed genes (p-value<0.05 and absolute ratio>0.7) based on Reactome databases using the geneSCF tool (32). The terms are considered significant only if enriched with p-value<0.05.

Statistics

All values are the means±SEM. Statistical analyses were performed using the Student's t test in Microsoft Excel. The p values are indicated. All categorical data used numbers and percentages. Quantitative data were presented using the median and range or mean. Differences between groups were evaluated using the chi square test for categorical variables and the Student's t test for continuous variables. Analyses were performed using SPSS 16.0 statistical software (SPSS Inc., Chicago, Ill.). All statistical tests were two-sided, and p-values<0.05 indicated statistical significance whereas p-values between 0.05 and 0.10 indicated a statistical tendency.

For Patients

The Student's t-test was used to compare continuous variables and chi-square test, or Fisher's exact test (when the conditions for use of the χ2-test were not fulfilled), were used for categorical variables. To guarantee the independence of primary cilium as a prognostic factor, the multivariate analysis was performed using Cox regression adjusted to the stage and age. DFS was defined as the time from surgery to the appearance of metastasis. OS was defined as the time between surgery and the date of death from any cause, censoring those alive at last follow-up. The Kaplan-Meier method was used to produce survival curves and analyses of censored data were performed using Cox models. All analyses were performed using R software, version 3.2.2 (Vienna, Austria, https://www.r-project.org/).

Results

Low VDAC1 and LGMN (Also Known as AEP) Expressions are Linked to Bad Prognosis in ccRCC Patients

By interacting with hexokinase, or members of the Bcl-2 family, VDAC1 supports glycolysis and prevents apoptosis. VDAC is thus involved in determining cellular survival or death, which is particularly relevant to cancer cells. To explore its possible role in ccRCC (Kidney renal clear cell carcinoma, KIRC), we first interrogated the Gene Expression Profiling Interactive Analysis (GEPIA) human dataset. Interestingly, the patients' overall survival (OS; Data not shown) and disease-free survival (DFS; Data not shown) plots revealed a direct correlation between low levels of VDAC1 and a poor prognosis. As we had previously shown a link between the asparagine endopeptidase (LGMN) and VDAC1 in hypoxia (Data not shown), we also explored the expression level of LGMN in the same cohort of ccRCC patients. Similar to VDAC1, both OS (Data not shown) and DFS (Data not shown) showed a strong correlation between low levels of LGMN and a poor prognosis. VDAC1 and LGMN expression was positively correlated in ccRCC patients (Data not shown), which enables us to explore the significance of this link in kidney cancer. We obtained 12 tissue samples of ccRCC patients from the Pathology Department of Nice (Data not shown Eight out of 12 (67%) were classified with a high expression of VDAC1 and the presence of VDAC1-ΔC (group A) whereas four out of 12 (33%) were classified with a low level of VDAC1 and the absence of VDAC1-ΔC (Data not shown). In group A, VDAC1-ΔC was present in tumor (T) tissues. In contrast, group B showed no VDAC1-ΔC in normal (N) or tumor (T) tissues. Finally, group A presented a higher LGMN expression level compared to group B (Data not shown), which correlates with the presence of VDAC1-ΔC (Data not shown). VDAC1-ΔC expression was also analyzed in different ccRCC cell lines. HK2, kidney epithelial cells from normal kidney, did not present VDAC1-ΔC under normoxic conditions (Data not shown). Analysis of VHL mutant RCC4 cell lines, in which the wild-type VHL gene has been restored (RCC4+pVHL), and thus mimicking normoxia with no stabilization of either HIF-1α or -2α isoforms, showed no VDAC1-ΔC whereas RCC4 cells with both stabilization of HIF-1α or -2α expressed VDAC1-ΔC. 786-O cells that only stabilize HIF-2a expressed the cleaved form of VDAC1 that was unexpectedly present even when pVHL was restored (786-O+pVHL). The presence of VDAC1-ΔC was also observed in RCC10 and A498-expressing HIF2-α ccRCC cell lines. These results suggested a strong link between VDAC1/VDAC1-ΔC and LGMN in the ccRCC context and described two groups of ccRCC patients with distinct prognoses. They also confirmed that VDAC1 cleavage is dependent on HIF-1 but independent on HIF-2, and revealed that HIF-2 may repress VDAC1 in a cell-dependent manner.

The Presence of VDAC1-ΔC in RCC4 and 786-O Cells Decreases or Abolishes Ciliation

We thus focused our research on ccRCC, which is associated with the loss of VHL function, deregulation of the hypoxia pathway and considered as a ciliopathy. We analyzed RCC4 and 786-O cell lines, VHL mutant cells, in which the wild-type gene has been restored (ccRCC+pVHL), for their ability to express the primary cilium depending on the presence or absence of VDAC1-ΔC. In comparison to RCC4+pVHL, which mimic normoxia with no stabilization of either HIF-1α or -2α, VHL mutant RCC4 cells stabilizing HIF-1α and -2α expressed VDAC1-ΔC (Data not shown). RCC4 showed a higher global metabolic phenotype compared to RCC4+pVHL cells (Data not shown). We next searched the presence of ciliated cells. We found that RCC4 cells had 50% fewer primary cilia compared to RCC4+pVHL in similar conditions of proliferation (Data not shown). Knockdown of HIF-α using siRNA against HIF-1α, or both HIF-1α and -2α, decreased the expression of VDAC1-ΔC and concomitantly increased the percentage of ciliated cells compared to siCtl (Data not shown). However, knockdown of HIF-2a alone had no effect on VDAC1-ΔC and the primary cilium. 786-O cells, only expressing HIF-2a, presented a cleaved form of VDAC1 in the absence or presence of functional pVHL (Data not shown). In these cells, absolutely no primary cilia were detected. Comparison of the metabolic phenotype of 786-O+pVHL cells, presenting VDAC1-ΔC, to RCC4+pVHL, with no VDAC1-ΔC, showed an increased metabolic phenotype in 786-O+pVHL compared to RCC4+pVHL cells (Data not shown). HIF-2α-targeted siRNA slightly increased the expression of VDAC1 and, consequently, the expression of VDAC1-ΔC, which in turn ensured the absence of ciliated cells (0%; Data not shown). VDAC1-ΔC and no ciliated cells were observed in two other HIF-2a expressing ccRCC cell lines, RCC10 and A498 (Data not shown). However, HIF-2α-targeted siRNA did not have an impact on VDAC1 in RCC10 and A498 cells and the absence of ciliated cells remained unchanged. These results reinforce the link between HIF-1, VDAC1-ΔC, tubulin and the primary cilium. These results demonstrated that VDAC1-ΔC controls resorption of the primary cilium in the HIF-1-dependent model of ccRCC but independently of HIF-2, and revealed that HIF-2 may repress VDAC1.

GLI1/IFT20 Signature is Correlated to Primary Cilium and VDAC1

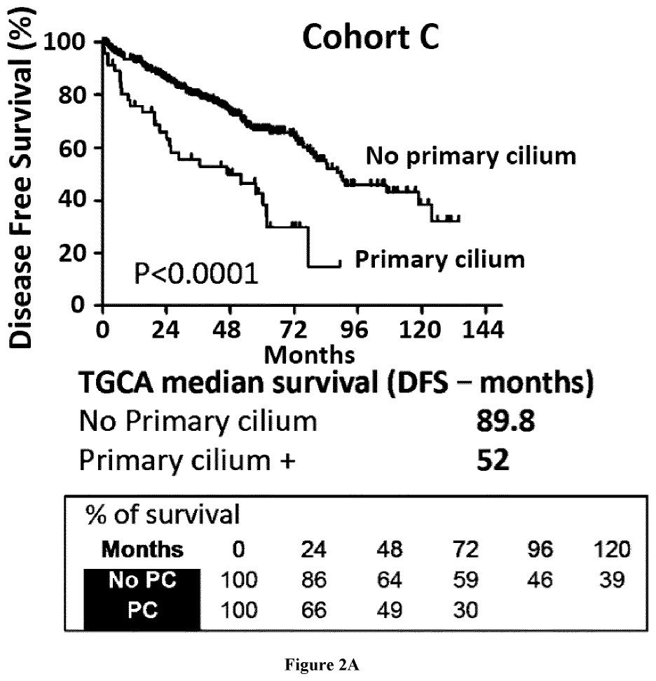

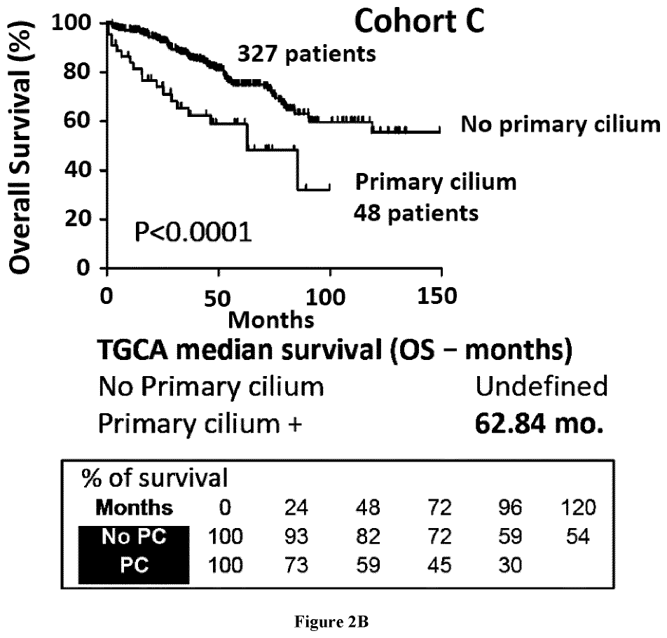

To further reinforce the link between VDAC1-ΔC and the percentage of ciliated cells, we evaluated the expression of genes implicated in the biogenesis and the activity of the primary cilium. The analysis of our transcriptomic data in Wt or Vdac1−/− MEF in hypoxia versus normoxia (22) highlighted differences in programs related to the primary cilium in the gene expression profile of both Wt and Vdac1−/− MEF (Data not shown). Moreover, we observed differences in gene expression profile between Wt and Vdac1−/− MEF suggesting that VDAC1 influenced genes are significantly associated with ciliogenesis. As mRNA levels of the GLI1 transcription factor (GLI1) and the intraflagellar transport protein 20 (IFT20) were shown to be modified (Data not shown), we choose to examine these two genes as read out for the activity and formation of the primary cilium. RCC4 cells presented a low expression level of both GLI1 and IFT20 and could be classified as GLI1−/IFT20− cells, in comparison to RCC4+pVHL (Data not shown). We then knocked down IFT20 and GLI1 in RCC4 cells (Data not shown). We observed increased expression of VDAC1 and, subsequently, an increase in the expression of VDAC1-ΔC, which slightly decreased the number of ciliated cells. In contrast, VDAC1-targeted siRNA increased the percentage of ciliated cells by more than 1.4-fold (Data not shown) and presented an expression profile of GLI1+ and IFT20+ cells compared to siRNA to Ctl (Data not shown). siRNA to VDAC1 cells were characterized by increased invasion than siRNA to control cells suggesting a more aggressive phenotype (Data not shown). Finally, on the basis of GLI1 and IFT20 expression, 786-O cells were considered to be GLI1−/IFT20+ compared to 786-O+pVHL cells (Data not shown). In order to block the hypoxic cleavage of VDAC1, the asparagine endopeptidase (AEP), that specifically cleaved VDAC1 at the aspargine 214, was silenced in RCC4 cells (Data not shown). VDAC1-ΔC totally disappeared (FIG. 2H), the cells acquired GLI1+/IFT20+ signature (Data not shown), expressed a higher percentage of primary cilium (FIG. 2K) and were more aggressive (Data not shown), similarly to what we have seen by downregulating VDAC1. These results demonstrated that downregulation of GLI1 and/or IFT20 expression correlated with the decrease or absence of primary cilia expression in ccRCC cells that showed an expression of VDAC1-ΔC. Moreover, decrease in VDAC1 expression was linked to more aggressiveness.

Absence of VDAC1-ΔC Promotes Aggressiveness in RCC4 but Allows Response to Antiglycolysis Treatments

Sunitinib, a vascular endothelial growth factor receptor inhibitor is widely used for patients with metastatic RCC. RCC4+pVHL−, RCC4− and RCC4 siVDAC1− cells were treated with 1.0 and 1.5 μM of sunitinib (Data not shown). Although all these cells were sensitive to sunitinib, we found that cells with VDAC1-ΔC were slightly more sensitive to treatment than RCC4+pVHL cells (with a GLI1+/IFT20+ signature and no VDAC1-ΔC) or cells with no VDAC1−ΔC or less VDAC1 (Data not shown). As RCC4 cells with VDAC1-ΔC present a higher glycolysis than RCC4+pVHL cells, we used 3 Bromopyruvate (3BP), an halogenated analogue of pyruvic acid that enters cells like a lactate via monocarboxylic acid transporters, to block glycolysis. Cells presenting no VDAC1-ΔC or less VDAC1 were highly sensitive to 10- and 25-μM of 3BP (Data not shown). Moreover, the aggressiveness of RCC4 siVDAC1 treated with 3BP was strongly decreased compared to control, (Data not shown).

All together these results suggest that sunitinib is not the best treatment for kidney cancer cells that present no VDAC1-ΔC. Our results propose a metabolic therapeutic approach, 3BP, which will be more efficient than the first line treatment already proposed.

Identification and Validation of the 5-Gene Signature Predictive of the Presence of VDAC1-ΔC and of the Primary Cilium of ccRCC Patients

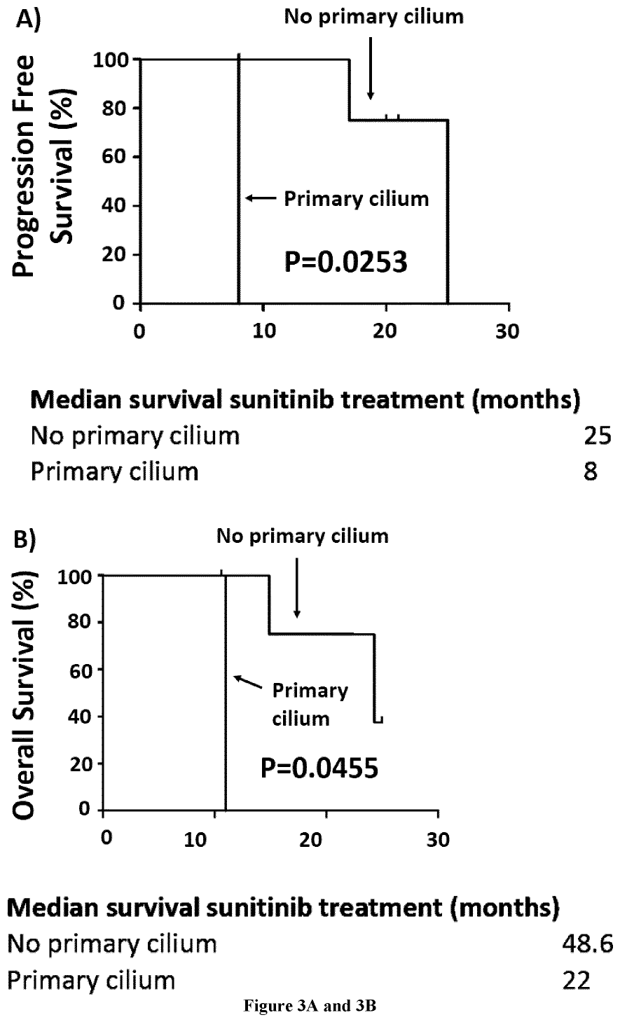

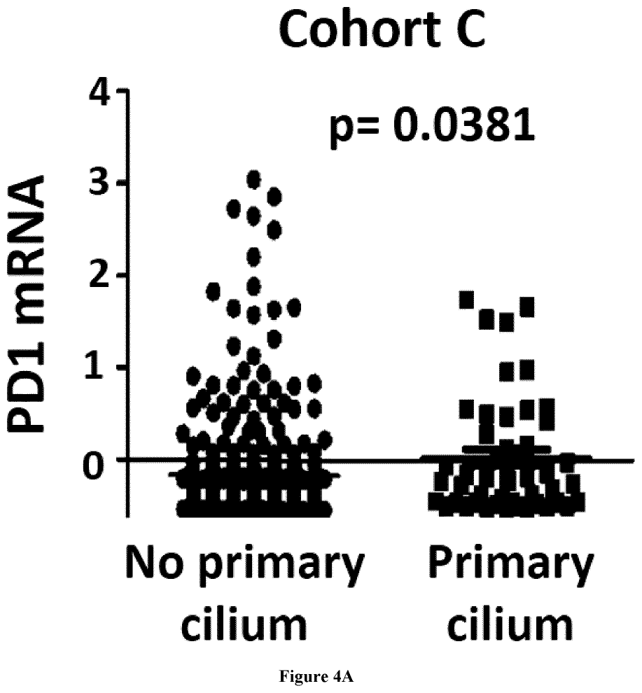

We obtained 12 tissue samples and slides of ccRCC patients from the Pathology Department of Nice (CHU) (Data not shown). To strengthen the GLI1 and IFT20 signature, other genes strongly modulated in hypoxia in Wt MEFs (Data not shown) were tested. From the different combinations used, we found that the association with the Platelet Derived Growth Factor Receptor a (PDGFRA), the Protein Kinase C Alpha (PRKCA) and the Frizzled Class receptor 1 (FZD1) genes was the most efficient in classifying patients (FIG. 3A). These five genes allowed the clustering of five groups: group 0 with two patients (GLI1+, IFT20−, PDGFRA−, PRKCA±, FZD1±), group 1 with two patients (GLI1−, IFT20−, PDGFRA−, PRKCA±, FZD1−), group 2 with 4 patients ((GLI1−, IFT20+, PDGFRA−, PRKCA±, FZD1−), group 3 with three patient (GLI1+, IFT20+, PDGFRA+, PRKCA+, FZD1−) and group 4 with one patient (GLI1+, IFT20+, PDGFRA−, PRKCA−, FZD1+). On the basis of GLI1 and IFT20 expression and our previous results in ccRCC cell lines, eight out of 12 (67%) were classified with a no primary cilium signature whereas four out of 12 (33%) were classified with a primary cilium signature. VDAC1-ΔC was present in groups 0, 1 and 2 (Data not shown). In group 2, VDAC1-ΔC was present in both normal (N) and tumor (T) tissues. In contrast, groups 3 and 4 showed no VDAC1-ΔC in normal (N) or tumor (T) tissues. Finally, we confirmed that the GLI1+/IFT20−, GLI1−/IFT20− or GLI1−/IFT20+ signatures were linked to the presence of VDAC1-ΔC and the absence of the primary cilium (0.3-3.7%), while the GLI1+/IFT20+ signature was linked to the absence of VDAC1-ΔC and the increased presence of the primary cilium (12-18%) (Data not shown). These results clearly demonstrate that this 5-gene expression signature provided a new type of classification according to the presence or absence of the primary cilium depending on VDAC1-ΔC. Moreover, our results unexpectedly show two groups of patients in which cancer cells express the primary cilium in a ciliopathic disease.

The Tumors of Primary Cilium Re-Expression Groups are More Aggressive than Tumors with Ciliopathy

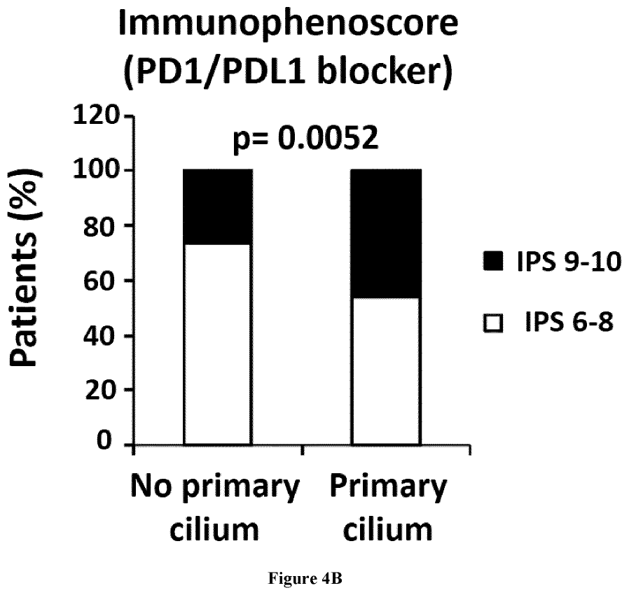

To investigate the possibility of predicting the prognosis of ccRCC patients based on the presence of the primary cilium, we assessed the ability of the five relevant gene signatures to cluster into the transcription profile of three cohorts. First, a cohort of patients from PREDIR (Cohort A) was used with 12 patients with hereditary pVHL mutations and 22 sporadic patients presenting with tumors with wild-type pVHL and mutated pVHL (Data not shown). Tissue samples from 43 patients with nonmetastatic ccRCC who had undergone surgery (Urology department of the Rennes University Hospital) (Cohort B) (Data not shown) (33) and 375 non-metatstatic ccRCC tumor samples produced by The Cancer Genome Atlas (TCGA) (Cohort C) (www.cbioportal.org, TCGA Provisional; RNA-Seq V2) (29) were analyzed. The same five groups with the same signature (Data not shown) were obtained. In Cohort A, 100% of the pVHL patients presented a signature with no primary cilium whereas two signatures of patients that should express the primary cilium were found in sporadic patients. No patient in the group 2 was found in this cohort. In the group 3, one (Cohort A)+four (Cohort B)+twenty (Cohort C) patients were identified, whereas in group 4, one (Cohort A)+five (Cohort B)+twenty-eight (Cohort C) patients were characterized. These groups represented a low percentage of patients with the primary cilium signature, 5.9%, 20.9% and 12.8% respectively. GLI1+/IFT20+ mRNA expression (primary cilium) correlated with shorter DFS (median survival of TCGA, 52 months versus 89.8 months (p 150 months), whereas group 2 presented a lower OS (118.8 months FIGS. 2A and 2B). We also established a correlation between the primary cilium and VDAC1 (Data not shown Patients with no primary cilium expressed a higher level of VDAC1 mRNA whereas patients with primary cilia expressed a low level of VDAC1 mRNA. Using the tumor proliferation marker KI67 (MKI67) to assess tumor growth, we observed no correlation (p=0.3573) between the absence or the presence of the primary cilium and proliferation, demonstrating that the presence of the primary cilium did not impact on the proliferation status of the tumor (Supplementary FIG. 4D). As expected, tumors with stabilization of HIF-2α only presented a tendency to be more aggressive (OS median survival: 72.38 months versus undefined, p=0.1035) compared to those expressing both HIF-1α/2α (Data not shown). A volcano plot analysis to further explore the difference between patients with a primary cilium signature and patients with no primary cilium signature showed 403 genes to be UP (1.8%) and 322 DOWN (1.5%) expressed when the mRNAs were significantly differentially expressed (Data not shown). The results of hierarchical cluster analyses showed distinguishable mRNA expression profiles between the primary cilium+ (PC+) patients and the no primary cilium (PC−) patients (Data not shown). Pathway analysis showed that the positively expressed mRNAs in these ccRCC patients were related to collagen biosynthesis and its modifying enzymes, ECM organization, collagen formation/degradation, degradation of the ECM, whereas the negatively expressed mRNAs were related to transport of small molecules, the solute-carrier-mediated transmembrane transport, metabolism of lipids, the TCA cycle. The UP mRNAs signature pathways strongly suggested involvement of EMT (Data not shown). However, the DOWN mRNAs referred to complete metabolic reprogramming. In depth analysis of the TCGA database revealed 310 patients with pVHL− ccRCC and 65 patients with pVHL+ ccRCC defined via deletions, mutations and promoter methylation in pVHL (Data not shown). Among the pVHL− tumors, 250 ccRCC tumors in the TCGA expressed both HIF-1 and -2 but 60 expressed only HIF-2. Using the primary cilium signature, we characterized two sub-groups, PC− and PC+ in each category. The PC+ group contained a low number of patients (10.4%, 21.7% and 13.8%) compared to the PC− group, and showed a lower median survival. We also found that the majority of PC+ patients were mostly at advanced tumor stages (stage 3/4—66%) rather than stage 1/2 (34%), whereas we observed the opposite with PC− patients, 25.2% and 74.8% respectively (Data not shown). Groups 3 and 4 of PC+ patients presented a more aggressive signature with an increase in processes reflecting extracellular matrix modifications coupled with a decrease in OXPHOS and lipid metabolism but maintenance of glycolysis, which favored epithelial-mesenchymal transition (EMT).

Finally, available in silico transcriptomic data showed that primary cilium analysis correlate with tumor stage and to a less extend with Furhman grade (Data not shown). Primary cilium signature, Furhman grade, tumor stage and age have an impact on DFS and OS (univariated analysis, Data not shown). Primary cilium signature represented a marker for DFS (Data not shown) and OS (Data not shown) independent of the tumor stage and age in a multivariate analysis. As an example, hazard ratios show that a PC+ patient will be twice as metastatic as PC− patients (Hazard ratio=2.448, DSF—Table 1C) and will decease faster (Hazard ratio=2.13, OS)—Data not shown). These results highlight that tumors from ccRCC patients expressing primary cilia with a GLI1+/IFT20+ signature but not VDAC1-ΔC are significantly more aggressive and characterized by a bad prognosis. In this context, the primary cilium is clearly a cancer promoter and maintenance of glycolysis seems to be crucial to support this aggressiveness.

The Tumors of Primary Cilium Re-Expression Groups have a Higher Score Indicative of Better Response to Immunotherapy and should Respond to Anti-Glycolysis Treatments

We previously showed that RCC4 cells expressing no VDAC1-ΔC are less sensitive to sunitinib but sensitive to 3BP. We thus checked if PC+ patients with a GLI1+/IFT20+ signature and no VDAC1-ΔC are resistant or sensitive to sunitinib. Analysis of PC+ patients from the clinical trial SUVEGIL (Clinical trials.gov Identifier: NCT00943839), treated with sunitinib, revealed that these patients had a lower survival than PC− groups (FIG. 3A and FIG. 3B) similar to what we obtained in RCC4 cells with siRNA to VDAC1 (Data not shown). Moreover, the PC+ group present a decrease in OXPHOS and lipid metabolism but maintenance of glycolysis suggesting a good chance of successful 3BP treatment for PC+ group of patients.

Finally, as immunotherapy has become increasingly common for the treatment of clear cell RCC, we investigated the immune profile for each group. Slight differences were observed in the relative fraction of major immune cell types in PC+ compared to PC− of the TCGA cohort (Data not shown) and a significant more proportions of T regulatory lymphocyte (Treg) was observed in PC+ group (Data not shown). To reinforce these results, PD1 mRNA expression was significantly increased in PC+ group (FIG. 4A) and immunophenoscore (FIG. 4B), used as a predictor of response to antiprogrammed cell death protein 1 (anti-PD1) was in favor of the PC+ group. These results clearly showed the immunotherapeutic potential specifically on the PC+.

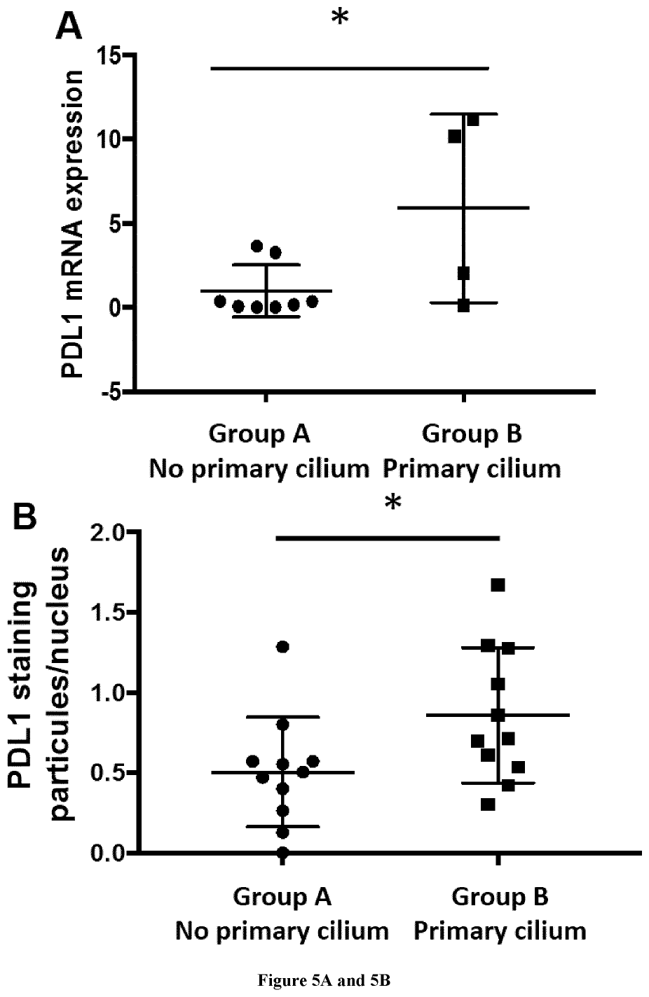

In the analysis of groups A (high level of VDAC1+VDAC1-ΔC) and B (low level of VDAC1 and absence of VDAC1-ΔC), we also observed a significant increase of PD-L1 mRNA expression in “primary cilium” patient group B (FIG. 5A) associated with an increased frequency of cytoplasmic PDL1 punctae in patient tumor sections within vesiclelike structures (FIG. 5B). These results clearly showed the specific immunotherapeutic potential for the “primary cilium” patient group.

All together these results suggest that sunitinib is not the best treatment for this specific PC+ group. Our results propose two therapeutic approaches, 3BP and/or anti-PD1, that could be used in first or second line only for the patients that present the strong PC+ signature, for which sunitinib is unfortunately less effective.

Discussion

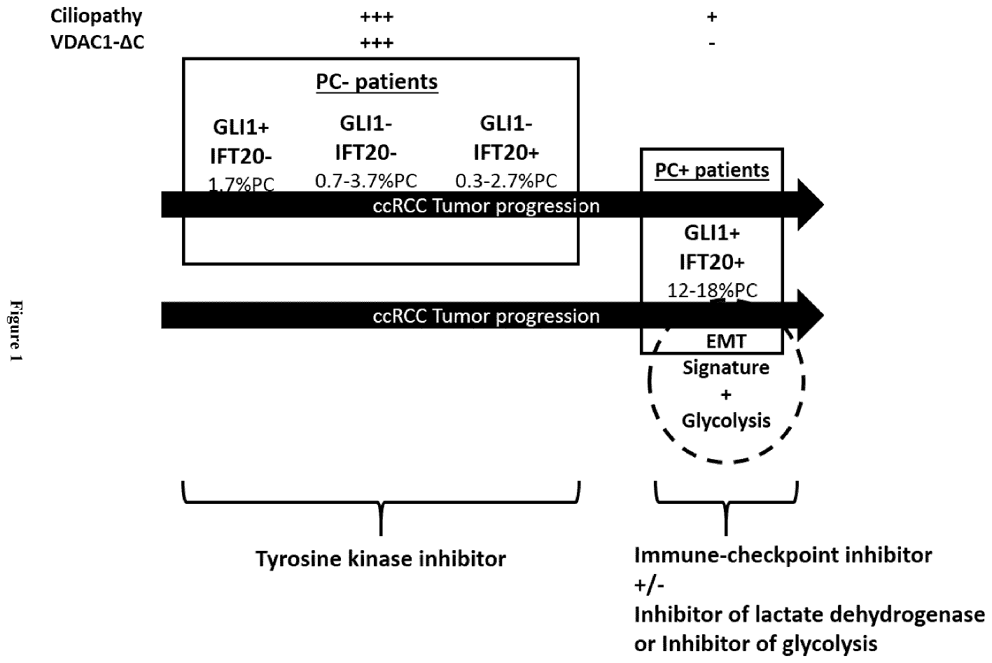

Our data describe a new function of control of ciliogenesis that is driven by VDAC1-AC, the form of VDAC1 that is produced in hypoxia. This novel function of VDAC1-4C-ciliopathy was characterized in ccRCC cells and in VHL and sporadic ccRCC patients (FIG. 1). The role of VDAC in metabolic homeostasis and cell death has been studied extensively (18,19,34-42). However, new functions for VDAC1 have recently been discovered. Rostovtseva et al. showed that the pore formed by VDAC can be regulated by dimeric tubulin, one of the most abundant cytoskeleton proteins (43,44). The interaction of tubulin acts as a plug inducing VDAC closure and thus controlling mitochondrial metabolism. Any modification to either tubulin or VDAC will modify their interaction, thus triggering important physiological events. Our findings reveal a different organization of tubulin within the cells depending on the presence or absence of VDAC1-ΔC (Data not shown). Compacted tubulin was observed around the nucleus in RCC4+pVHL cells (without VDAC1-ΔC), in comparison with RCC4 cells (presenting VDAC1-ΔC) in which tubulin appeared diffuse. Only diffuse tubulin was observed in 786-O+pVHL and 786-O cells, both characterized by the cleavage of VDAC1. Due to this, we speculate that the presence of VDAC1-ΔC may impair ciliogenesis by shifting the balance towards an unpolimerized state of tubulin and by inhibiting the VDAC1/tubulin interaction, thus causing changes in cellular metabolism. Analyses are already in process. Moreover, a role of importance to VDAC has been attributed; that of controlling ciliogenesis (23). Majumder et al. recently showed that centrosomal VDAC3 associated with the centrosome via Mps1, a protein kinase that plays a role in centriole assembly (23). The Mps1-VDAC3 complex, and also centrosomal VDAC1, were involved in negative regulation of ciliogenesis. However, the cell model used by Majumder et al. concerned retinal cells rather than cancer cell models and we studied mitochondrial VDAC1 instead of centrosomal VDAC1. Our preliminary results have shown that VDAC1 is in close proximity to the centrosome (Data not shown) suggesting that VDAC1 could directly participate in ciliogenesis, a mechanism that we are exploring further. Since 2012, we have constantly investigated the role of VDAC1-ΔC in hypoxia (21, 22, 45) and under iron deprivation conditions (46). Our study describes, for the first time, a VDAC1-ΔC dependent mechanism in which kidney cancer cells can maintain glycolysis in the presence of EMT signature, which promotes survival of cells surrounded by an unfavorable microenvironment. Our study strongly shows that the 5-gene expression signature we characterized is closely related to the formation of VDAC1-ΔC and the absence of the primary cilium. The global OS allowed us to classify patients from those with the less aggressive to the more aggressive tumors. However, we also described a new group (groups 3 and 4: GLI1+/IFT20+) of patients expressing/re-expressing the primary cilium in a ciliopathy context. We demonstrated that patients belonging to this group had much more aggressive tumors than patients with little or no primary cilia. Although this group expressed fewer primary cilia (from 12 to 18%) than healthy tissue (from 50 to 80%), it had a 5-gene expression signature close to the group of healthy tissue. These results suggested that this group could be directly derived from healthy tissue but that the expression of the EMT genes made the tumors of these patients more aggressive. In the first cohort (Cohort A-PREDIR) we studied, the GLI1+/IFT20+ signature was observed only in sporadic patients, suggesting that such expression/re-expression was impossible in patients with VHL mutations at the germinal level. However, the cohort of VHL patients was too small to draw conclusions. Interestingly, we also found that each group (pVHL−/HIF-1+/HIF-2+, pVHL−/HIF-2+ and pVHL+) from the TCGA database presented 10.4, 21.7 and 13.8% of patients, respectively, with a GLI1+/IFT20+ signature. The median survival of these patients was lower than for patients with a no primary cilium signature. Moreover, patients with this GLI1+/IFT20+ signature presented a significant correlation between aggressiveness and a lower VDAC1. Similarly, in our study, siRNA to VDAC1 and AEP in RCC4 cells, that shifted the signature in GLI1+/IFT20+, were characterized by increased migration and invasion than siRNA to control cells suggesting a more aggressive phenotype. The tumor aggressiveness in these patients could be the combination of a switch to EMT process activation, re-expression or maintenance of the primary primary cilium, together with a decrease in VDAC expression and thus a disappearance of the cleaved form of VDAC1.

In patients who progressively lose the primary cilium, this loss generated increased metabolism and greater resistance to cell death, promoting tumor growth. Since this loss was related to the presence of the cleaved form of VDAC1, the strategy would therefore be to prevent the cleavage of VDAC1 to restore the presence of the primary cilium. Combination of siRNA to VDAC1 or AEP with 3BP increased cell death in RCC4 cells. However, in patients who express/re-express the primary cilium and since this group expressed high levels of KI67 indicating that they are highly proliferative, it is unlikely that the primary cilium is the only force associated with aggressiveness. Indeed, we characterized a signature related to EMT that can explain the aggressive phenotype of the tumors of these patients. However, remodeling of metabolism was changed. Although cancer metabolism is a hallmark of cancer, it has been shown that aberrant metabolism supports EMT (47,48). In the signature of the present study, lipid metabolism and the TCA signature were downregulated in correlation with observations made by Hakimi et al. for ccRCC patients (49). However, expression of glycolytic enzymes was not modified, strongly suggesting that this is the metabolic pathway that the cancer cells of patients in groups 3 and 4 favored. It is therefore possible to envisage specific treatment for these groups: Temsirolimus (50) or Everolimus, specific inhibitors of mTOR (51) that block proliferation, in combination with small molecule inhibitors that prevent EMT such as EW-7197 or IN-1130, through a block in TGFβ1 and 2, have already been used in metastatic breast and lung cancer (52). By maintaining only one metabolic pathway, cancer cells with a GLI1+/IFT20+ signature offer a metabolic vulnerability that it would be wise to exploit. We have shown that inhibitors of glycolysis such as 3-bromopyruvate, used as proof-of-concept, or inhibitors of lactate production (dichloroacetate, FX11, AZD-3965) (48) are of interest. Finally, combination of an immune checkpoint inhibitor such as atezolizumab or nivolumab (an anti-PDL1 or PD1 inhibitor) alone or plus 3-bromopyruvate could be evaluated on PC+ patients with such reduced overall survival (FIG. 1). Indeed, the presence of PD-L1 in group B strongly suggests an important role in promoting tumor progression. We have proposed several hypotheses to explain such expression patterns. Firstly, Noman and Chouaib have revealed the binding of HIF-1a to PD-L1 promoter (53). However, we showed that the presence of primary cilia in patients from group 4 was independent of HIF-1, HIF-2 or pVHL. Secondly, it has been reported that PDL1 works predominantly in lactate-enriched tumor microenvironments (54). As tumors of patients from group 4 maintain glycolysis, and thus lactate production, this suitable microenvironment may protect cancer cells and thus could participate in the activation of PD-L1. Thirdly, epigenetic regulation was revealed to be involved in PD-L1 expression in cancer cells (55). MiRs, P53 and also STAT3 were reported to regulate PD-L1 expression. As ccRCC patients are mainly p53 wild type, we focused on STAT3 and observed a significant (p<0.001) increase of STAT3 expression in patients from group 4 (data not shown). Understanding what regulates PDL1 in patients expressing primary cilium will be thus an important avenue of research going forward. This novel classification of GLI1+/IFT20+ ccRCC patients should have an impact on clinical practice, not only in characterizing new subgroups of ccRCC patients, but also in offering new combinations of treatments that are much more effective and more specific for a specific group of ccRCC patients.

### REFERENCES

Throughout this application, various references describe the state of the art to which this invention pertains. The disclosures of these references are hereby incorporated by reference into the present disclosure.
1. Fliegauf M, Benzing T, Omran H. When cilia go bad: cilia defects and ciliopathies. Nat Rev Mol Cell Biol 2007; 8(11):880-93 doi 10.1038/nrm2278.
2. Gradilone S A, Pisarello M J L, LaRusso N F. Primary Cilia in Tumor Biology: The Primary Cilium as a Therapeutic Target in Cholangiocarcinoma. Current drug targets 2017; 18(8):958-63 doi 10.2174/1389450116666150223162737.
3. Michaud E J, Yoder B K. The primary cilium in cell signaling and cancer. Cancer Res 2006; 66(13):6463-7 doi 10.1158/0008-5472.CAN-06-0462.
4. Hassounah N B, Bunch T A, McDermott K M. Molecular pathways: the role of primary cilia in cancer progression and therapeutics with a focus on Hedgehog signaling. Clin Cancer Res 2012; 18(9):2429-35 doi 10.1158/1078-0432.CCR-11-0755.
5. Gerhardt C, Leu T, Lier J M, Ruther U. The cilia-regulated proteasome and its role in the development of ciliopathies and cancer. Cilia 2016; 5:14 doi 10.1186/s13630-016-0035-3.
6. Badano J L, Mitsuma N, Beales P L, Katsanis N. The ciliopathies: an emerging class of human genetic disorders. Annual review of genomics and human genetics 2006; 7:125-48 doi 10.1146/annurev.genom.7.080505.115610.
7. Gerdes J M, Davis E E, Katsanis N. The vertebrate primary cilium in development, homeostasis, and disease. Cell 2009; 137(1):32-45 doi 10.1016/j.cell.2009.03.023.
8. Hynes M, Stone D M, Dowd M, Pitts-Meek S, Goddard A, Gurney A, et al. Control of cell pattern in the neural tube by the zinc finger transcription factor and oncogene Gli-1.

Neuron 1997; 19(1):15-26.
9. Ruiz I Altaba A. Gli proteins encode context-dependent positive and negative functions: implications for development and disease. Development 1999; 126:3205-16.
10. Berbari N F, O'Connor A K, Haycraft C J, Yoder B K. The primary cilium as a complex signaling center. Current biology: CB 2009; 19(13):R526-35 doi 10.1016/j.cub.2009.05.025.
11. Fry A M, Leaper M J, Bayliss R. The primary cilium: guardian of organ development and homeostasis. Organogenesis 2014; 10(1):62-8 doi 10.4161/org.28910.
12. Plotnikova O V, Golemis E A, Pugacheva E N. Cell cycle-dependent ciliogenesis and cancer. Cancer Res 2008; 68(7):2058-61 doi 10.1158/0008-5472.CAN-07-5838.
13. Thoma C R, Frew I J, Krek W. The VHL tumor suppressor: riding tandem with GSK3beta in primary cilium maintenance. Cell Cycle 2007; 6(15):1809-13 doi 10.4161/cc.6.15.4518.
14. Chapman J R, Wong G. Cancer in patients with inherited ciliopathies: polycystic kidney disease. The Lancet Oncology 2016; 17(10):1343-5 doi 10.1016/S1470-2045(16)30307-2.
15. Richard S, Gardie B, Couve S, Gad S. Von Hippel-Lindau: how a rare disease illuminates cancer biology. Semin Cancer Biol 2013; 23(1):26-37 doi 10.1016/j.semcancer.2012.05.005.
16. Esteban M A, Harten S K, Tran M G, Maxwell P H. Formation of primary cilia in the renal epithelium is regulated by the von Hippel-Lindau tumor suppressor protein. J Am Soc Nephrol 2006; 17(7):1801-6 doi 10.1681/ASN.2006020181.
17. Brahimi-Horn M, Laferrière J, Mazure N, Pouysségur J. Hypoxia and tumour progression. In: Marmé D, Fusenig N, editors. Tumor Angiogenesis. Springer ed: Springer; 2007. p 171-94.
18. Shoshan-Barmatz V, Kmita H, Lemasters J J. Editorial: Uncovering the Function of the Mitochondrial Protein VDAC in Health and Disease: From Structure-Function to Novel Therapeutic Strategies. Front Oncol 2017; 7:320 doi 10.3389/fonc.2017.00320.
19. Shoshan-Barmatz V, Krelin Y, Shteinfer-Kuzmine A, Arif T. Voltage-Dependent Anion Channel 1 As an Emerging Drug Target for Novel Anti-Cancer Therapeutics. Front Oncol 2017; 7:154 doi 10.3389/fonc.2017.00154.
20. Chiche J, Rouleau M, Gounon P, Brahimi-Horn M C, Pouyssegur J, Mazure N M. Hypoxic enlarged mitochondria protect cancer cells from apoptotic stimuli. Journal of cellular physiology 2010; 222(3):648-57 doi 10.1002/jcp.21984.
21. Brahimi-Horn M C, Ben-Hail D, Ilie M, Gounon P, Rouleau M, Hofman V, et al. Expression of a truncated active form of VDAC1 in lung cancer associates with hypoxic cell survival and correlates with progression to chemotherapy resistance. Cancer research 2012; 72(8):2140-50 doi 10.1158/0008-5472.CAN11-3940.
22. Brahimi-Horn M C, Giuliano S, Saland E, Lacas-Gervais S, Sheiko T, Pelletier J, et al. Knockout of Vdac1 activates hypoxia-inducible factor through reactive oxygen species generation and induces tumor growth by promoting metabolic reprogramming and inflammation. Cancer Metab 2015; 3:8 doi 10.1186/s40170-015-0133-5.
23. Majumder S, Cash A, Fisk H A. Non-Overlapping Distributions and Functions of the VDAC Family in Ciliogenesis. Cells 2015; 4(3):331-53 doi 10.3390/cells4030331.
24. Majumder S, Slabodnick M, Pike A, Marquardt J, Fisk H A. VDAC3 regulates centriole assembly by targeting Mps1 to centrosomes. Cell Cycle 2012; 11(19):3666-78 doi 10.4161/cc.21927.
25. Richard D E, Berra E, Gothie E, Roux D, Pouyssegur J. p42/p44 mitogen-activated protein kinases phosphorylate hypoxia-inducible factor lalpha (HIF-lalpha) and enhance the transcriptional activity of HIF-1. J Biol Chem 1999; 274(46):32631-7.
26. Bellot G, Garcia-Medina R, Gounon P, Chiche J, Roux D, Pouyssegur J, et al. Hypoxia-induced autophagy is mediated through hypoxia-inducible factor induction of BNIP3 and BNIP3L via their BH3 domains. Molecular and cellular biology 2009; 29(10):2570-81 doi 10.1128/MCB.00166-09.
27. Bilton R, Mazure N, Trottier E, Hattab M, Dery M A, Richard D E, et al. Arrestdefective-1 protein, an acetyltransferase, does not alter stability of hypoxiainducible factor (HIF)-1alpha and is not induced by hypoxia or HIF. J Biol Chem 2005; 280(35):31132-40.
28. Gattoliat C-H, Couvé S, Meurice G, Oréar C, Droin N, Chiquet M, et al. Integrative analysis of dysregulated microRNAs and mRNAs in multiple recurrent synchronized renal tumors from patients with von Hippel-Lindau disease. Int J Oncol 2018.
29. Dufies M, Nollet M, Ambrosetti D, Traboulsi W, Viotti J, Borchiellini D, et al. Soluble C D146 is a predictive marker of pejorative evolution and of sunitinib efficacy in clear cell renal cell carcinoma. Theranostics 2018; 8(9):2447-58 doi 10.7150/thno.23002.
30. Chen F, Zhang Y, Senbabaoglu Y, Ciriello G, Yang L, Reznik E, et al. Multilevel Genomics-Based Taxonomy of Renal Cell Carcinoma. Cell Rep 2016; 14(10):2476-89 doi 10.1016/j.celrep.2016.02.024.
31. Ricketts C J, De Cubas A A, Fan H, Smith C C, Lang M, Reznik E, et al. The Cancer Genome Atlas Comprehensive Molecular Characterization of Renal Cell Carcinoma. Cell Rep 2018; 23(12):3698 doi 10.1016/j.celrep.2018.06.032.
32. Subhash S, Kanduri C. GeneSCF: a real-time based functional enrichment tool with support for multiple organisms. BMC bioinformatics 2016; 17(1):365 doi 10.1186/s12859-016-1250-z.
33. Ambrosetti D, Dufies M, Dadone B, Durand M, Borchiellini D, Amiel J, et al. The two glycolytic markers GLUT1 and MCT1 correlate with tumor grade and survival in clear-cell renal cell carcinoma. PLoS One 2018; 13(2):e0193477 doi 10.1371/journal.pone.0193477.
34. Colombini M. Structure and mode of action of a voltage dependent anionselective channel (VDAC) located in the outer mitochondrial membrane. Annals of the New York Academy of Sciences 1980; 341:552-63.
35. De Pinto V, Tomasello F, Messina A, Guarino F, Benz R, La Mendola D, et al. Determination of the conformation of the human VDAC1 N-terminal peptide, a protein moiety essential for the functional properties of the pore. Chembiochem 2007; 8(7):744-56 doi 10.1002/cbic.200700009.
36. Lemasters J J, Holmuhamedov E. Voltage-dependent anion channel (VDAC) as mitochondrial governator—thinking outside the box. Biochimica et biophysica acta 2006; 1762(2):181-90 doi 10.1016/j.bbadis.2005.10.006.
37. Maldonado E N, Sheldon K L, DeHart D N, Patnaik J, Manevich Y, Townsend D M, et al. Voltage-dependent anion channels modulate mitochondrial metabolism in cancer cells: regulation by free tubulin and erastin. J Biol Chem 2013; 288(17):11920-9 doi 10.1074/jbc.M112.433847.
38. Pastorino J G, Hoek J B. Regulation of hexokinase binding to VDAC. J Bioenerg Biomembr 2008; 40(3):171-82 doi 10.1007/s10863-008-9148-8.
39. Shoshan-Barmatz V, De S, Meir A. The Mitochondrial Voltage-Dependent Anion Channel 1, Ca(2+) Transport, Apoptosis, and Their Regulation. Front Oncol 2017; 7:60 doi 10.3389/fonc.2017.00060.
40. Vander Heiden M G, Chandel N S, Schumacker P T, Thompson C B. Bcl-xL prevents cell death following growth factor withdrawal by facilitating mitochondrial ATP/ADP exchange. Mol Cell 1999; 3(2):159-67.
41. Wenner C E. Targeting mitochondria as a therapeutic target in cancer. Journal of cellular physiology 2012; 227(2):450-6 doi 10.1002/jcp.22788.
42. Zalk R, Israelson A, Garty E S, Azoulay-Zohar H, Shoshan-Barmatz V. Oligomeric states of the voltage-dependent anion channel and cytochrome c release from mitochondria. The Biochemical journal 2005; 386(Pt 1):73-83 doi 10.1042/BJ20041356.
43. Rostovtseva T K, Bezrukov S M. VDAC inhibition by tubulin and its physiological implications. Biochim Biophys Acta 2012; 1818(6):1526-35 doi 10.1016/j.bbamem.2011.11.004.
44. Rostovtseva T K, Gurney P A, Hoogerheide D P, Rovini A, Sirajuddin M, Bezrukov S M. Sequence diversity of tubulin isotypes in regulation of the mitochondrial voltage-dependent anion channel. J Biol Chem 2018; 293(28):10949-62 doi 10.1074/jbc.RA117.001569.
45. Brahimi-Horn M C, Lacas-Gervais S, Adaixo R, Tic K, Rouleau M, Notte A, et al. Local mitochondrial-endolysosomal microfusion cleaves the voltage-dependent anion channel 1 to promote survival in hypoxia. Molecular and cellular biology 2015; 35(9):1491-505 doi 10.1128/MCB.01402-14.
46. Ferecatu I, Canal F, Fabbri L, Mazure N M, Bouton C, Golinelli-Cohen M P. Dysfunction in the mitochondrial Fe—S assembly machinery leads to formation of the chemoresistant truncated VDAC1 isoform without HIF-lalpha activation. PLoS One 2018; 13(3):e0194782 doi 10.1371/journal.pone.0194782.
47. Huang R, Zong X. Aberrant cancer metabolism in epithelial-mesenchymal transition and cancer metastasis: Mechanisms in cancer progression. Crit Rev Oncol Hematol 2017; 115:13-22 doi 10.1016/j.critrevonc.2017.04.005.
48. Sciacovelli M, Frezza C. Metabolic reprogramming and epithelial-to-mesenchymal transition in cancer. Febs J 2017; 284(19):3132-44 doi 10.1111/febs.14090.
49. Hakimi A A, Reznik E, Lee C H, Creighton C J, Brannon A R, Luna A, et al. An Integrated Metabolic Atlas of Clear Cell Renal Cell Carcinoma. Cancer Cell 2016; 29(1):104-16 doi 10.1016/j.ccell.2015.12.004.
50. Meric-Bernstam F, Mills G B. Mammalian target of rapamycin. Seminars in oncology 2004; 31(6 Suppl 16):10-7; discussion 33.
51. Motzer R J, Escudier B, Oudard S, Hutson T E, Porta C, Bracarda S, et al. Efficacy of everolimus in advanced renal cell carcinoma: a double-blind, randomised, placebo-controlled phase III trial. Lancet 2008; 372(9637):449-56 doi 10.1016/S0140-6736(08)61039-9.
52. Kothari A N, Mi Z, Zapf M, Kuo P C. Novel clinical therapeutics targeting the epithelial to mesenchymal transition. Clinical and translational medicine 2014; 3:35 doi 10.1186/s40169-014-0035-0.
53. Noman M Z, Chouaib S. Targeting hypoxia at the forefront of anticancer immune responses. Oncoimmunology. 2014; 3: e954463.
54. Feng J, Yang H, Zhang Y, Wei H, Zhu Z, Zhu B, et al. Tumor cell-derived lactate induces TAZ-dependent upregulation of PD-L1 through GPR81 in human lung cancer cells. Oncogene. 2017; 36: 5829-39.
55. Wang Y, Wang H, Yao H, Li C, Fang J Y, Xu J. Regulation of PD-L1: Emerging Routes for Targeting Tumor Immune Evasion. Frontiers in pharmacology. 2018; 9: 536.

1. A method for selecting a cancer treatment in treating a subject suffering from a cancer, wherein said method comprises the step of:
(i) determining in a biological sample obtained from said subject the expression level of GLI family zinc finger 1 (GLI1) and intraflagellar transport 20 (IFT20)
(ii) comparing the expression levels quantified at step i) with their predetermined references values and
(iii) concluding that the subject will be treated with at least one therapeutic agent whose target is linked to metabolism and/or immunotherapy, when the expression level of GLI1 is higher than its predetermined reference value 1 (PRV1) and if the expression level of IFT20 is higher than its predetermined reference value 2 (PRV2).

2. A method for selecting a cancer treatment in a subject suffering from a cancer, wherein said method comprises the step of:
(i) determining in a biological sample obtained from said subject the expression level of GLI family zinc finger 1 (GLI1) and intraflagellar transport 20 (IFT20)
(ii) comparing the expression levels quantified at step i) with their predetermined references values and
(iii) concluding that the subject will be treated with a current treatment, if:
a) the expression level of GLI1 is lower than its PRV1 and if the expression level of IFT20 is higher than its PRV2, or
b) the expression level of GLI1 is higher than its PRV1 and if the expression level of IFT20 is lower than its PRV2, or
c) the expression level of GLI1 is lower than its PRV1 and if the expression level of IFT20 is lower than its PRV2.

3. The method according to claim 1, wherein the subject is treated with an immune checkpoint inhibitor in combination with an inhibitor of lactate dehydrogenase (LDH) or an inhibitor of glycolysis.

4. A method for treating a subject suffering from a cancer, comprising:
(i) determining in a biological sample obtained from said subject the expression level of GLI1 and IFT20
(ii) comparing the expression levels quantified at step i) with their predetermined references values and
(iii) administering to said subject a therapeutically effective amount of at least one therapeutic agent whose target is linked to metabolism and/or immunotherapy, when the expression level of GLI1 is higher than its predetermined reference value 1 (PRV1) and the expression the expression level of IFT20 is higher than its predetermined reference value 2 (PRV2),
wherein the at least one therapeutic agent is an immune checkpoint inhibitor, an inhibitor of lactate dehydrogenase (LDH) or an inhibitor of glycolysis.

5. A method for treating a subject suffering from a cancer, comprising:
(i) treating the subject with a agent whose target is linked to metabolism and/or immunotherapy,
(ii) determining in a biological sample obtained from said subject the expression level of GLI1 and IFT20,
(iii) comparing the expression levels quantified at step (ii) with their predetermined references values and
(iv) administering to said subject a therapeutically effective amount of the agent when: the expression level of GLI1 is lower than its PRV1 and the expression level of IFT20 is higher than its PRV2, or
a) the expression level of GLI1 is higher than its PRV1 and the expression level of IFT20 is lower than its PRV2, or
b) the expression level of GLI1 is lower than its PRV1 and the expression level of IFT20 is lower than its PRV2.

6. The method according to claim 4, wherein step (iii) comprises administering to said subject a therapeutically effective amount of an immune checkpoint inhibitor in combination with an inhibitor of lactate dehydrogenase (LDH) or an inhibitor of glycolysis.

7. The method according to claim 4, wherein the cancer is a ciliopathy.

8. The method according to claim 4, wherein the cancer is a kidney cancer.

9. The method according to claim 8 wherein, the kidney cancer is a Clear cell renal cell carcinoma (ccRCC).

10. The method according to claim 5, wherein the tyrosine kinase inhibitor is sunitinib.

11. The method according to claim 4, wherein the immune checkpoint inhibitor is anti-PD-L1 or anti-PD-1.

12. The method according to claim 4, wherein the immune checkpoint inhibitor is atezolizumab.

13. The method according to claim 4, wherein the immune checkpoint inhibitor nivolumab.

14. The method according to claim 4, wherein the inhibitor of lactate dehydrogenase (LDH) is selected from dichloroacetate (DCA), FX11, and AZD-3965.

15. The method according to claim 4, wherein the inhibitor of glycolysis is 3-bromopyruvate.

16. The method of claim 1, wherein the agent is an immune checkpoint inhibitor, an inhibitor of lactate dehydrogenase (LDH) or an inhibitor of glycolysis.

17. The method of claim 5, wherein the agent is a tyrosine kinase inhibitor.
